(19) United States
(12) Patent Application Publication
(10) Pub. No.: US 2011/0190386 A1
(43) Pub. Date: Aug. 4, 2011

(54) Aptamer Inhibitors of Osteopontin and Methods of Use Thereof

(76) Inventors: Paul C. Kuo, Chapel Hill, NC (US); Zhiyong Mi, Durham, NC (US); Hongtao Guo, Chapel Hill, NC (US)

(21) Appl. No.: 12/867,063

(22) PCT Filed: Feb. 11, 2009
(86) PCT No.: PCT/US09/00868

§ 371 Date: Apr. 14, 2011

Related U.S. Application Data
(60) Provisional application No. 61/027,592, filed on Feb. 11, 2008.

(51) Int. Cl. A61K 31/7088; C12N 15/63; C12N 5/10; G01N 33/68; C07H 21/00; C07H 21/02; C12N 1/00; C12N 5/09; A61P 35/00; A61P 35/04; A61P 43/00

(52) U.S. Cl. 514/44 R; 435/320.1; 435/419; 435/325; 436/501; 536/23.1; 435/243; 435/375

(57) ABSTRACT

The present invention polynucleotide aptamers that selectively bind to and inhibit the function of osteopontin, pharmaceutical compositions comprising the same, and methods of use for diagnostics and treatment of diseases and disorders associated with osteopontin.

### FIELD OF THE INVENTION

The present invention polynucleotide aptamers that selectively bind to and inhibit the function of osteopontin, pharmaceutical compositions comprising the same, and methods of use for diagnostics and treatment of diseases and disorders associated with osteopontin.

### BACKGROUND OF THE INVENTION

Cancer progression depends on an accumulation of metastasis-supporting physiological changes which are regulated by cell signaling molecules. One such molecule, osteopontin (OPN), is a secreted phosphoprotein which functions as a cell attachment protein and cytokine that signals through two cell adhesion molecules: αᵥβ₃-integrin and CD44 (Denhardt et al., Ann. NY Acad. Sci. 760:127 (1995); Denhardt et al., Annu. Rev. Pharmacol. Toxicol. 41:723 (2001); Weber et al., Proc. Assoc. Am Physicians 109:1 (1997)). Initially discovered as an inducible tumor-promoter gene, OPN is an acidic hydrophilic glycophosphoprotein which is overexpressed in human tumors and is the major phosphoprotein secreted by malignant cells in advanced metastatic cancer (Denhardt et al., J. Cell Biochem. 56:48 (1994); Brown et al., Am. J. Pathol. 145:610 (1994); Agrawal et al., J. Natl. Cancer Inst. 94:513 (2002); Coppola et al., Clin. Cancer Res. 10:184 (2004); Das et al., J. Biol. Chem. 278:28593 (2003); Fedarko et al., Clin. Cancer Res. 7:4060 (2001); Gotoh et al., Pathol. Int. 52:19 (2002); Grano et al., J. Biol. Regul. Homeost. Agents 16:190 (2002)). Evidence has accumulated for involvement of OPN in increased cellular migratory and invasive behavior, increased metastasis, protection from apoptosis, promotion of colony formation and 3D growth ability, induction of tumor-associated inflammatory cells, and induction of expression of angiogenic factors (Tuck et al., J. Cell Biochem. 102:859 (2007)). Gain- and loss-of-function assays have demonstrated a critical role for OPN in tumor metastatic function in colon, liver, and breast cancers (Wai et al., J. Surg. Res. 121:228 (2004)). Clinical studies have clearly linked serum OPN expression with increased metastatic tumor burden and poor patient outcomes (Coppola et al., Clin. Cancer Res. 10:184 (2004); Mazar et al., Angiogenesis. 3:15 (1999)).

OPN was initially characterized in 1979 as a phosphoprotein secreted by transformed, malignant epithelial cells (Senger et al., Cell 16:885 (1979)). It is a member of the small integrin-binding ligand N-linked glycoprotein (SIBLING) family of proteins which include bone sialoprotein (BSP), dentin matrix protein 1 (DMP1), dentin sialoprotein (DSPP), and matrix extracellular phosphoglycoprotein (MEPE) (Fedarko et al., FASEB J. 18:734 (2004)). The molecular structure of OPN is rich in aspartate and sialic-acid residues and contains unique functional domains which mediate critical cell-matrix and cell-cell signaling through the αᵥβ₃ integrin and CD44 receptors in a variety of normal and pathologic processes. Integrin αᵥβ₃ is detected consistently in breast cancer bone-metastases and αᵥβ₃ contributes to metastatic behavior in several ways (Liapis et al., Diagn. Mol. Pathol. 5:127 (1996)). OPN-integrin binding directly mediates migration and invasion of tumor cells, enhances endothelial cell migration, survival and lumen formation during angiogenesis, represents a downstream target for vascular endothelial growth factor signaling in microvascular endothelial cells with a direct role in angiogenesis, activates osteoclasts in lytic bone metastases, and alters host-immunity by increasing interleukin (IL)-12 expression in murine macrophages and interferon expression in natural killer cells (Wai et al., Carcinogenesis 26:741 (2005); Rangaswami et al., Trends Cell Biol. 16:79 (2006)). CD44 variants, especially CD44v6, have been identified as protein markers for metastatic behavior in hepatocellular, breast, lung, pancreatic, colorectal and gastric cancers and in lymphomas (Goodison et al., Mol. Pathol. 52:189 (1999); Ponta et al., Nat. Rev. Mol. Cell Biol. 4:33 (2003)). OPN can interact specifically with CD44v6 and/or v7 (Goodison et al., Mol. Pathol. 52:189 (1999); Ponta et al., Nat. Rev. Mol. Cell Biol. 4:33 (2003); Gao et al., Carcinogenesis 24:1871 (2003)). CD44v7-10 ligation of OPN mediates chemotaxis and adhesion of fibroblasts, T-cells and bone marrow cells, downregulates the host-inflammatory response in an IL-10 mediated manner, and confers metastatic potential when overexpressed through plasmid vectors models of pancreatic cancer. Binding of OPN with αᵥβ₃ integrin upregulates plasma membrane expression of CD44v6 and augments in vitro adhesion of HepG2 hepatocellular carcinoma cells (Gao et al., Carcinogenesis 24:1871 (2003)). Lin et al have demonstrated that increased survival and growth of IL-3 dependent mouse bone marrow cells is mediated by OPN, and CD44 antibody attenuates these effects (Lin et al., Mol. Cell Biol. 20:2734 (2000)). Studies also suggest that OPN and CD44 interact with the ezrin, radixin and moesin (ERM) proteins to alter cytoskeletal dynamics, cell adhesion, and motility through the cortical actin filaments (Zohar et al., J. Cell Physiol. 184:118 (2000); Zohar et al., Eur. J. Oral Sci. 106 Suppl 1:401 (1998)).

A critical component of tumorigenesis and metastasis is the degradation of the basement membrane and interstitial matrix by MMPs and uPA, as part of the plasminogen-activator-plasmin system. MMPs are extracellular matrix-degrading enzymes that play a crucial role in embryogenesis, tissue remodeling, inflammation and angiogenesis. MMP2 and MMP9 are important contributors to the process of invasion, tumor growth and metastasis. Both MMP2 and MMP9 efficiently degrade native type IV and V collagens, fibronectin, ectactin and elastin (Deryugina et al., Cancer Metastasis Rev. 25:9 (2006); Overall et al., Nat. Rev. Cancer 6:227 (2006)). Studies have shown a correlation between MMP2 activation and metastatic potential (Deryugina et al., Cancer Metastasis Rev. 25:9 (2006); Overall et al., Nat. Rev. Cancer 6:227 (2006)). uPA, its receptor uPAR, and inhibitors PAI-1 and PAI-2, which together constitute the uPA system, play a vital role in not only cancer progression but also in several normal physiological processes such as wound healing, liver regeneration and homeostasis (Das et al., IUBMB. Life 57:441 (2005); Durand et al., Thromb. Haemost. 91:438 (2004); Pillay et al., Trends Biotechnol. 25:33 (2007)). High levels of uPA are associated with cancers of the lung, skin, breast, bladder, uterine cervix and soft tissue sarcoma (Pillay et al., Trends Biotechnol. 25:33 (2007)). uPA interacts with uPAR to facilitate the conversion of plasminogen into the widely acting serine protease plasmin, which regulates cell invasion by degrading matrix proteins such as type IV collagen, gelatin, fibronectin and laminin or acts indirectly by activating MMPs. Studies indicate that blocking of uPA activity or the uPA-uPAR interaction drastically downregulates tumor growth and metastasis (Bauer et al., Cancer Res. 65:7775 (2005); Mi et al., Carcinogenesis 27:1134 (2006)).

OPN appears to regulate the activity of at least two ECM-degrading proteins. Philip et al demonstrate that OPN upregulates pro-MMP2 expression in a NF-κB-dependent fashion during extracellular matrix invasion (Philip et al., J. Biol. Chem. 278:14487 (2003)). Transfection of IκBα abrogates OPN-induced MMP expression while MMP2 antisense oligonucleotides reduce OPN-mediated migration and ECM invasion in B16F10 murine melanoma cells. A novel function of the thrombin-cleaved COOH-terminal fragment of OPN has recently been described (Mi et al., Cancer Res. 67:4088 (2007)). This fragment binds cyclophilin C to the CD147 cell surface receptor to activate Akt1/2 and MMP2 to enhance matricellular proteolysis. OPN also increases cell invasiveness in human mammary carcinoma through stimulation of uPA (Tuck et al., Breast Cancer Res. Treat. 70:197 (2001)). The uPA system is elevated in breast cancer patients with poor prognosis, in malignant cancers and in bone metastases (Mi et al., Carcinogenesis 27:1134 (2006); Andreasen et al., Int. J. Cancer 72:1 (1997)). Das et al have confirmed that OPN induction of uPA depends on PI 3′-kinase/Akt activity (Das et al., J. Biol. Chem. 278:28593 (2003); Das et al., IUBMB. Life 57:441 (2005)). It has been demonstrated that OPN upregulates uPA and MMP2 activity through integrin-linked kinase (ILK) and AP-1 signaling during tumor cell invasion (Mi et al., Carcinogenesis 27:1134 (2006)). Together, these studies indicate that OPN activates MMP and uPA through a variety of overlapping signaling pathways.

With regard to OPN signaling, Kundu's group has demonstrated that OPN induces PI3K activity and PI3K-dependent Akt phosphorylation through the αᵥβ₃ integrin-mediated pathway in breast cancer cells (Das et al., J. Biol. Chem. 278:28593 (2003); Das et al., J. Biol. Chem. 279:11051 (2004)). In addition, overexpression of PTEN, a phosphatase that can antagonize PI3K signaling, suppresses OPN-induced Akt activation during osteoclast differentiation and cell motility (Rangaswami et al., Trends Cell Biol. 16:79 (2006)). OPN-CD44 interactions promote cell survival and motility through activation of PI3K-dependent pathways. OPN also stimulates Src-dependent AP-1 activation, regulates negative crosstalk between NIK/ERK and MEKK1/JNK1 pathways, and activates the mitogen-activated protein kinase pathway (Rangaswami et al., Trends Cell Biol. 16:79 (2006)). All of these elements contribute to cancer cell motility, invasion, tumor growth and metastasis. As a secreted phosphoprotein that is readily accessible in the extracellular milieu, OPN is an attractive therapeutic target for blockade of metastasis.

In addition to a role in cancer and metastasis, OPN plays a role in other diseases and disorders, including inflammatory and immune disorders. OPN is involved in restenosis of arteries through the ongoing processes of local inflammation, thrombosis, and smooth muscle cell migration and proliferation. OPN is also involved in immunity to infectious diseases due to its ability to costimulate T cell proliferation, enhance interferon-γ and IL-12 production, and diminish IL-10 production.

Methods of treating cancer, restenosis, autoimmune diseases, bone diseases, and other disorders by inhibiting the expression or function of OPN using antibodies (U.S. Pat. Nos. 7,241,873; 7,282,490; U.S. Published Application No. 2006/0263383) or nucleic acids that bind to OPN mRNA (U.S. Pat. No. 6,458,590; U.S. Published Application Nos. 2006/0252684; 2004/0142865) have been described.

Recently, small structured single-stranded RNAs, also known as RNA aptamers, have emerged as viable alternatives to small-molecule and antibody-based therapy (Que-Gewirth et al., Gene Ther. 14:283 (2007); Ireson et al., Mol. Cancer Ther. 5:2957 (2006)). RNA aptamers specifically bind target proteins with high affinity, are quite stable, lack immunogenicity, and elicit biological responses. Aptamers are evolved by means of an iterative selection method called SELEX (systematic evolution of ligands by exponential enrichment) to specifically recognize and tightly bind their targets by means of well-defined complementary three-dimensional structures.

RNA aptamers represent a unique emerging class of therapeutic agents (Que-Gewirth et al., Gene Ther. 14:283 (2007); Ireson et al., Mol. Cancer Ther. 5:2957 (2006)). They are relatively short (12-30 nucleotide) single-stranded RNA oligonucleotides that assume a stable three-dimensional shape to tightly and specifically bind selected protein targets to elicit a biological response. In contrast to antisense oligonucleotides, RNA aptamers can effectively target extracellular targets, such as OPN. Like antibodies, aptamers possess binding affinities in the low nanomolar to picomolar range. In addition, aptamers are heat stable, lack immunogenicity, and possess minimal interbatch variability. Chemical modifications, such as amino or fluoro substitutions at the 2′ position of pyrimidines, may reduce degradation by nucleases. The biodistribution and clearance of aptamers can also be altered by chemical addition of moieties such as polyethylene glycol and cholesterol. Further, SELEX allows selection from libraries consisting of up to 10¹⁵ ligands to generate high-affinity oligonucleotide ligands to purified biochemical targets, such as OPN. Recently, the aptamer pegaptanib was approved for the treatment of age-related macular degeneration (Wong et al., Lancet 370:204 (2007)). With regard to the field of oncology, the DNA aptamer GBI-10, derived from a human glioblastoma cell line, was recently demonstrated to bind tenascin-C (Daniels et al., Proc. Natl. Acad. Sci. USA 100:15416 (2003)). Similarly, RNA aptamers have been demonstrated to target the Ku DNA repair proteins with resulting sensitization of breast cancer cells to etoposide (Zhang et al., Int. J. Mol. Med. 14:153 (2004)). As a secreted protein, OPN represents an ideal target for RNA aptamer mediated inhibition.

### SUMMARY OF THE INVENTION

The present invention relates to polynucleotide aptamers that specifically bind to OPN and block the binding of osteopontin to its cognate receptors, CD44 and integrin. OPN is an attractive target for inhibition by aptamers as it is a secreted protein that is readily accessible in the extracellular matrix. By blocking the ability of OPN to bind to its receptors and stimulate downstream pathways, the aptamers inhibit the ability of OPN to stimulate the adhesion, migration, and or invasion characteristics of cells comprising the receptors, thereby inhibiting the metastasis potential of the cells. Thus, one aspect of the present invention relates to polynucleotide aptamers that specifically bind to OPN. In one embodiment, the aptamers are DNA or RNA aptamers or hybrid DNA/RNA aptamers. In another embodiment, the OPN is human and/or mouse OPN. In a further embodiment, the aptamer comprises the sequence of any one of SEQ ID NOS: 1-14. Another aspect of the invention relates to polynucleotides encoding the aptamers of the invention, vectors comprising the polynucleotides, and cells comprising the polynucleotides. A further aspect of the invention relates to pharmaceutical compositions comprising the aptamers of the invention.

One aspect of the present invention relates to methods of using the aptamers of the invention to inhibit OPN function. One embodiment relates to methods of inhibiting at least one function of OPN, comprising contacting OPN with the aptamers of the present invention. Another embodiment relates to methods of inhibiting binding of OPN to CD44 and/or integrin receptors, comprising contacting OPN with the aptamers of the invention. A further embodiment relates to methods of inhibiting the adhesion, migration, and/or invasion ability of a cell, comprising CD44 and/or integrin receptors, comprising contacting the cells with the aptamers of the present invention. Another embodiment relates to methods of treating diseases and disorders associated with OPN in a subject, comprising administering to the subject the aptamers of the invention. OPN-associated diseases and disorders include, without limitation, cancer, metastasis, autoimmune disorders, inflammatory disorders, bone disorders, and restenosis. An additional embodiment relates to methods of treating cancer in a subject, comprising administering to the subject the aptamers of the invention. Another embodiment relates to methods of inhibiting tumor metastasis in a subject, comprising administering to the subject the aptamers of the invention. A further embodiment relates to methods of promoting wound healing and preventing scar formation in a subject, comprising administering to the subject the aptamers of the invention.

Another aspect of the invention relates to methods of using the aptamers of the invention for diagnostic purposes, e.g., measuring levels of OPN or binding of OPN to its receptors and diagnosing diseases and disorders related to OPN.

The present invention is explained in greater detail in the drawings herein and the specification set forth below.

### BRIEF DESCRIPTION OF THE DRAWINGS

FIG. 1A shows the OPN-R3 aptamer (SEQ ID NO: 1) secondary structure model. The theoretical structure was determined by the mFold program at www.idtdna.com/Scitools/Applications/mFold/.

FIG. 1B shows RNA electrophoretic mobility shift assays of OPN-R3. RNA aptamer (OPN-R3) was synthesized and end-labeled with [γ-³²P] ATP. The reactions were resolved and visualized by autoradiography. In specific competitive binding assays, unlabeled OPN-R3 type aptamers were added at a 20-fold molar excess. In nonspecific competitive binding assays, unlabeled mutant aptamer was used. Supershift assays were performed by preincubating recombinant human OPN with rabbit anti human OPN polyclonal antibody (Santa Cruz Biotechnology). The blot is representative of four experiments.

FIG. 1C shows mutant OPN RNA aptamers (OPN-R3-1 (SEQ ID NO: 17), OPN-R3-2(SEQ ID NO: 18), OPN-R3-3(SEQ ID NO: 19)).

FIG. 1D shows RNA electrophoretic mobility shift assays of OPN-R3-1, OPN-R3-2, and OPN-R3-3. Mutant RNA aptamers were synthesized and end-labeled with [γ-³²P] ATP. The reactions were resolved and visualized by autoradiography. In specific competitive binding assays, unlabeled OPN-R3 and OPN-R3-1 aptamers were added at a 20-fold molar excess. The blot is representative of four experiments.

FIG. 2A shows Western blot analysis of OPN expression in MDA-MB231 cell lysate and culture medium. Cells were lysed in buffer and protein concentration was determined by the Bio-Rad protein assay kit; the protein samples were separated by 4-20% SDS-PAGE and electrotransferred onto polyvinylidene difluoride membranes by semi-dry transfer. The membranes were probed with the primary antibodies for OPN and β-actin. These antibodies were detected using the appropriate horseradish peroxidase-conjugated secondary antibody. The reactive proteins were visualized by means of chemiluminescence. The blot is representative of three experiments. (N/A, not applicable.)

FIG. 2B shows FRET analysis of MDA-MB231 cells. Human full length CD44s cDNA and hOPN-a cDNA were separately fused in frame into mammalian expression vector pECFP and pEYFP, respectively. MDA cells were then transfected with both plasmids. CFP and YFP emission spectra were collected following excitation at 458 nm and were used as reference spectra for linear unmixing of CFP and YFP emission spectra. FRET was measured by acceptor photobleaching. FRET was measured as an increase in CFP fluorescence intensity following YFP photobleaching. FRET efficiency was calculated as 100×[(CFP post-bleach−CFP prebleach)/CFP post-bleach]; FRET efficiency was measured and calculated by Leica LAS AF software. FRET was performed on 50 cells per treatment group with 3 regions per cell. Photos are representative of 5 experiments.

FIG. 3A shows Western blot analysis of PI3K, JNK1/2, Src, Akt, MMP2 and uPA in MDA-MB231 cells. Cells were lysed in buffer and protein concentration was determined by the Bio-Rad protein assay kit; the protein samples were separated by 4-20% SDS-PAGE and electrotransferred onto polyvinylidene difluoride membranes by semi-dry transfer. The membranes were probed with the appropriate primary antibodies. These antibodies were detected using the appropriate horseradish peroxidase-conjugated secondary antibody. The reactive proteins were visualized by means of chemiluminescence. The blot is representative of four experiments.

FIG. 3B shows a histogram of PI3K and P-JNK1/2 expression. PI3K and P-JNK1/2 are normalized to β-actin and total JNK expression, respectively, by laser densitometry. Data are presented as mean ±SEM of four experiments. (*, p<0.01 vs. No treatment, OPN, Mutant OPN-R3, CD44 Ab, OPN-R3+RNase; #, p<0.01 vs. No treatment, OPN, αᵥβ₃ Ab, Mutant OPN-R3, CD44 Ab, OPN-R3+RNase; **, p<0.01 vs. No treatment, OPN, Mutant OPN-R3, OPN-R3, OPN-R3+RNase; @, p<0.01 vs. No treatment, OPN, Mutant OPN-R3, OPN-R3, OPN-R3+RNase).

FIG. 3C shows a histogram of P-Src and P-Akt expression. P-Src and P-Akt are normalized to Total Src and Total Akt expression, respectively, by laser densitometry. Data are presented as mean±SEM of four experiments. (*, p<0.01 vs. No treatment, OPN, Mutant OPN-R3, CD44 Ab, OPN-R3+RNase; #, p<0.01 vs. No treatment, OPN, αᵥβ₃ Ab, Mutant OPN-R3, CD44 Ab, OPN-R3+RNase; **, p<0.01 vs. No treatment, OPN, Mutant OPN-R3, OPN-R3, OPN-R3+RNase; @, p<0.01 vs. No treatment, OPN, Mutant OPN-R3, OPN-R3, OPN-R3+RNase).

FIG. 3D shows a histogram of MMP2 and uPA expression. MMP2 and uPA are normalized to β-actin expression by laser densitometry. Data are presented as mean±SEM of four experiments. (*, p<0.02 vs. No treatment, OPN, Mutant OPN-R3, OPN-R3+RNase; #, p<0.01 vs. αᵥβ₃ Ab, CD44 Ab).

FIG. 4 shows adhesion, migration and invasion characteristics of MDA-MB231 cells. In vitro adhesion, migration and invasion assays were performed. Data are presented as mean±SEM of four experiments. (*, p<0.01 vs. No treatment, OPN, αᵥβ₃ Ab, Mutant OPN-R3, CD44 Ab, OPN-R3+RNase; #, p<0.01 vs. No treatment, OPN, Mutant OPN-R3, OPN-R3, OPN-R3+RNase).

FIG. 5A shows the mean bioluminescence of MDA-MB231 cells at the primary tumor site. Photos are representative of four animals in each group. *P<0.01 day 20 modified OPN-R3 vs. mutant OPN-R3 and no treatment; **P<0.01 day 30 modified OPN-R3 vs. mutant OPN-R3 and no treatment.

FIG. 5B shows volume of primary tumors. Tumor volume (V) is calculated using the following formula: V=(1/2)S²×L (S, shortest dimension; L, longest dimension). All data are presented as mean±SD (n=4 per treatment group). *P<0.01 day 20 modified OPN-R3 vs. mutant OPN-R3 and no treatment.

FIG. 5C shows mean bioluminescence of MDA-MB231 cells metastatic to the lung. Photos are representative of four animals in each group. *P<0.01 lung-modified OPN-R3 vs. mutant OPN-R3 and no treatment; **P<0.01 primary tumor-modified OPN-R3 vs. mutant OPN-R3 and no treatment.

FIG. 6A shows microarray heat map analysis of mouse primary tumors treated with OPN-R3 (left), wild-type non-treatment (middle), and mutant OPN-R3 aptamer. The panel shows gene expression fold change compared with the mean normalized value of controls (wild-type non-treatment and mutant OPN-R3 aptamer treatment).

FIGS. 6B-6C show scatter plots shows differentially expressed genes between mutant OPN-R3 aptamer treatment and OPN-R3 aptamer treatment (B) and between wild-type non-treatment and mutant OPN-R3 treatment control (C).

FIG. 6D shows a list of the dysregulated genes associated with down-regulated and up-regulated canonical signal transduction pathways.

FIG. 7A shows four down-regulated canonical biochemical and molecular biology pathways with significant (p<0.05, Fisher's exact test) correlation in comparison to the wild-type non-treatment and mutant OPN-R3 aptamer treatment controls.

FIG. 7B shows four up-regulated canonical biochemical and molecular biology pathways with significant (p<0.05, Fisher's exact test) correlation in comparison to the wild-type non-treatment and mutant OPN-R3 aptamer treatment controls.

FIG. 8A shows a histogram of mRNA changes in MDA-MB231 primary tumor from animals treated with OPN-R3 or mutant OPN-R3.

FIG. 8B shows Western blots of differentially expressed proteins in MDA-MB231 primary tumor from animals treated with OPN-R3 or mutant OPN-R3.

### DETAILED DESCRIPTION OF PREFERRED EMBODIMENTS

### Definitions.

The teen “isolated” designates a biological material (nucleic acid or protein) that has been removed from its original environment (the environment in which it is naturally present). For example, a polynucleotide present in the natural state in a plant or an animal is not isolated, however the same polynucleotide separated from the adjacent nucleic acids in which it is naturally present, is considered “isolated”. The term “purified” does not require the material to be present in a form exhibiting absolute purity, exclusive of the presence of other compounds. It is rather a relative definition.

A “nucleic acid” or “polynucleotide” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester anologs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

The term “fragment” will be understood to mean a nucleotide sequence of reduced length relative to the reference nucleic acid and comprising, over the common portion, a nucleotide sequence identical to the reference nucleic acid. Such a nucleic acid fragment according to the invention may be, where appropriate, included in a larger polynucleotide of which it is a constituent. Such fragments comprise, or alternatively consist of, oligonucleotides ranging in length from at least 6, 8, 9, 10, 12, 15, 18, 20, 21, 22, 23, 24, 25, 30, 39, 40, 42, 45, 48, 50, 51, 54, 57, 60, 63, 66, 70, 75, 78, 80, 90, 100, 105, 120, 135, 150, 200, 300, 500, 720, 900, 1000 or 1500 consecutive nucleotides of a nucleic acid according to the invention.

Several methods known in the art may be used to propagate a polynucleotide according to the invention. Once a suitable host system and growth conditions are established, recombinant expression vectors can be propagated and prepared in quantity. As described herein, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

A “vector” is any means for the cloning of and/or transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. The term “vector” includes both viral and nonviral means for introducing the nucleic acid into a cell in vitro, ex vivo or in vivo. A large number of vectors known in the art may be used to manipulate nucleic acids, incorporate response elements and promoters into genes, etc. Possible vectors include, for example, plasmids or modified viruses including, for example bacteriophages such as lambda derivatives, or plasmids such as pBR322 or pUC plasmid derivatives, or the Bluescript vector. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified or any site may be produced by ligating nucleotide sequences (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

Viral vectors, and particularly retroviral vectors, have been used in a wide variety of gene delivery applications in cells, as well as living animal subjects. Viral vectors that can be used include but are not limited to retrovirus, adeno-associated virus, pox, baculovirus, vaccinia, herpes simplex, Epstein-Barr, adenovirus, geminivirus, and caulimovirus vectors. Non-viral vectors include plasmids, liposomes, electrically charged lipids (cytofectins), DNA-protein complexes, and biopolymers. In addition to a nucleic acid, a vector may also comprise one or more regulatory regions, and/or selectable markers useful in selecting, measuring, and monitoring nucleic acid transfer results (transfer to which tissues, duration of expression, etc.).

Vectors may be introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., J. Biol. Chem. 267:963 (1992); Wu et al., J. Biol. Chem. 263:14621 (1988); and Hartmut et al., Canadian Patent Application No. 2,012,311, filed Mar. 15, 1990).

A polynucleotide according to the invention can also be introduced in vivo by lipofection. For the past decade, there has been increasing use of liposomes for encapsulation and transfection of nucleic acids in vitro. Synthetic cationic lipids designed to limit the difficulties and dangers encountered with liposome-mediated transfection can be used to prepare liposomes for in vivo transfection of a gene encoding a marker (Feigner et al., Proc. Natl. Acad. Sci. USA 84:7413 (1987); Mackey, et al., Proc. Natl. Acad. Sci. U.S.A. 85:8027 (1988); and Ulmer et al., Science 259:1745 (1993)). The use of cationic lipids may promote encapsulation of negatively charged nucleic acids, and also promote fusion with negatively charged cell membranes (Feigner et al., Science 337:387 (1989)). Particularly useful lipid compounds and compositions for transfer of nucleic acids are described in International Patent Publications WO95/18863 and WO96/17823, and in U.S. Pat. No. 5,459,127. The use of lipofection to introduce exogenous genes into the specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney, and the brain. Lipids may be chemically coupled to other molecules for the purpose of targeting (Mackey, et al., 1988, supra). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931).

It is also possible to introduce a vector in vivo as a naked DNA plasmid (see U.S. Pat. Nos. 5,693,622, 5,589,466 and 5,580,859). Receptor-mediated DNA delivery approaches can also be used (Curiel et al., Hum. Gene Ther. 3:147 (1992); Wu et al., J. Biol. Chem. 262:4429 (1987)).

The term “transfection” means the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths may have identical promoter activity.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

“Transcriptional and translational control sequences” are DNA regulatory sequences, such as promoters, enhancers, terminators, and the like, that provide for the expression of a coding sequence in a host cell. In eukaryotic cells, polyadenylation signals are control sequences.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The plasmids or vectors may further comprise at least one promoter suitable for driving expression of a gene in a host cell. The term “expression vector” means a vector, plasmid or vehicle designed to enable the expression of an inserted nucleic acid sequence following transformation into the host. The cloned gene, i.e., the inserted nucleic acid sequence, is usually placed under the control of control elements such as a promoter, a minimal promoter, an enhancer, or the like. Initiation control regions or promoters, which are useful to drive expression of a nucleic acid in the desired host cell are numerous and familiar to those skilled in the art. Virtually any promoter capable of driving these genes is suitable for the present invention including but not limited to: viral promoters, bacterial promoters, animal promoters, mammalian promoters, synthetic promoters, constitutive promoters, tissue specific promoter, developmental specific promoters, inducible promoters, light regulated promoters; CYC1, HIS3, GAL1, GAL4, GAL10, ADH1, PGK, PHO5, GAPDH, ADC1, TRP1, URA3, LEU2, ENO, TPI, alkaline phosphatase promoters (useful for expression in Saccharomyces); AOX1 promoter (useful for expression in Pichia); β-lactamase, lac, ara, tet, trp, IP_{L}, IP_{R}, T7, tac, and trc promoters (useful for expression in Escherichia coli); light regulated-, seed specific-, pollen specific-, ovary specific-, pathogenesis or disease related-, cauliflower mosaic virus 35S, CMV 35S minimal, cassaya vein mosaic virus (CsVMV), chlorophyll a/b binding protein, ribulose 1,5-bisphosphate carboxylase, shoot-specific, root specific, chitinase, stress inducible, rice tungro bacilliform virus, plant super-promoter, potato leucine aminopeptidase, nitrate reductase, mannopine synthase, nopaline synthase, ubiquitin, zein protein, and anthocyanin promoters (useful for expression in plant cells); animal and mammalian promoters known in the art include, but are not limited to, the SV40 early (SV40e) promoter region, the promoter contained in the 3′ long terminal repeat (LTR) of Rous sarcoma virus (RSV), the promoters of the E1A or major late promoter (MLP) genes of adenoviruses (Ad), the cytomegalovirus (CMV) early promoter, the herpes simplex virus (HSV) thymidine kinase (TK) promoter, a baculovirus IE1 promoter, an elongation factor 1 alpha (EF1) promoter, a phosphoglycerate kinase (PGK) promoter, a ubiquitin (Ubc) promoter, an albumin promoter, the regulatory sequences of the mouse metallothionein-L promoter and transcriptional control regions, the ubiquitous promoters (HPRT, vimentin, α-actin, tubulin and the like), the promoters of the intermediate filaments (desmin, neurofilaments, keratin, GFAP, and the like), the promoters of therapeutic genes (of the MDR, CFTR or factor VIII type, and the like), pathogenesis or disease related-promoters, and promoters that exhibit tissue specificity and have been utilized in transgenic animals, such as the elastase I gene control region which is active in pancreatic acinar cells; insulin gene control region active in pancreatic beta cells, immunoglobulin gene control region active in lymphoid cells, mouse mammary tumor virus control region active in testicular, breast, lymphoid and mast cells; albumin gene, Apo AI and Apo AII control regions active in liver, alpha-fetoprotein gene control region active in liver, alpha 1-antitrypsin gene control region active in the liver, beta-globin gene control region active in myeloid cells, myelin basic protein gene control region active in oligodendrocyte cells in the brain, myosin light chain-2 gene control region active in skeletal muscle, and gonadotropic releasing hormone gene control region active in the hypothalamus, pyruvate kinase promoter, villin promoter, promoter of the fatty acid binding intestinal protein, promoter of the smooth muscle cell α-actin, and the like. In addition, these expression sequences may be modified by addition of enhancer or regulatory sequences and the like.

Enhancers that may be used in embodiments of the invention include but are not limited to: an SV40 enhancer, a cytomegalovirus (CMV) enhancer, an elongation factor I (EF1) enhancer, yeast enhancers, viral gene enhancers, and the like.

Termination control regions, i.e., terminator or polyadenylation sequences, may also be derived from various genes native to the preferred hosts. Optionally, a termination site may be unnecessary, however, it is most preferred if included. In a preferred embodiment of the invention, the termination control region may be comprise or be derived from a synthetic sequence, synthetic polyadenylation signal, an SV40 late polyadenylation signal, an SV40 polyadenylation signal, a bovine growth hormone (BGH) polyadenylation signal, viral terminator sequences, or the like.

The terms “3′ non-coding sequences” or “3′ untranslated region (UTR)” refer to DNA sequences located downstream (3′) of a coding sequence and may comprise polyadenylation [poly(A)] recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“Regulatory region” means a nucleic acid sequence that regulates the expression of a second nucleic acid sequence. A regulatory region may include sequences which are naturally responsible for expressing a particular nucleic acid (a homologous region) or may include sequences of a different origin that are responsible for expressing different proteins or even synthetic proteins (a heterologous region). In particular, the sequences can be sequences of prokaryotic, eukaryotic, or viral genes or derived sequences that stimulate or repress transcription of a gene in a specific or non-specific manner and in an inducible or non-inducible manner. Regulatory regions include origins of replication, RNA splice sites, promoters, enhancers, transcriptional termination sequences, and signal sequences which direct the polypeptide into the secretory pathways of the target cell.

The term “percent identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences may be performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method may be selected: KTUPLE 1, GAP PENALTY=3, WINDOW=5 and DIAGONALS SAVED=5.

The term “sequence analysis software” refers to any computer algorithm or software program that is useful for the analysis of nucleotide or amino acid sequences. “Sequence analysis software” may be commercially available or independently developed. Typical sequence analysis software will include but is not limited to the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990), and DNASTAR (DNASTAR, Inc. 1228 S. Park St. Madison, Wis. 53715 USA). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized.

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of cancer, a therapeutically effective amount preferably refers to the amount of a therapeutic agent that decreases the rate of tumor growth, decreases tumor mass, decreases the number of metastases, increases time to tumor progression, or increases survival time by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The terms “prevent,” “preventing,” and “prevention,” as used herein, refer to a decrease in the occurrence of pathological cells (e.g., hyperproliferative or neoplastic cells) in an animal. The prevention may be complete, e.g., the total absence of pathological cells in a subject. The prevention may also be partial, such that the occurrence of pathological cells in a subject is less than that which would have occurred without the present invention.

The term “treat,” as used herein, refers to any type of treatment that imparts a benefit to a patient afflicted with a disease or disorder, including improvement in the condition of the patient (e.g., in one or more symptoms), delay in the progression of the disease, etc.

“Cancer,” as used herein, may be any type of cancer, including but not limited to breast cancers; osteosarcomas; angiosarcomas; fibrosarcomas and other sarcomas; leukemias; lymphomas; sinus tumors; ovarian, uretal, bladder, prostate and other genitourinary cancers; colon, esophageal, and stomach cancers and other gastrointestinal cancers; lung cancers; myelomas; pancreatic cancers; liver cancers; kidney cancers; endocrine cancers; skin cancers; and brain or central and peripheral nervous (CNS) system tumors, including gliomas and neuroblastomas.

“Pharmaceutically acceptable,” as used herein, means that the compound or composition is suitable for administration to a subject to achieve the treatments described herein, without unduly deleterious side effects in light of the severity of the disease and necessity of the treatment.

The term “specifically binds,” as used herein, refers to a molecule (e.g., an aptamer) that binds to a target (e.g., a protein) with at least five-fold greater affinity as compared to any non-targets, e.g., at least 10-, 20-, 50-, or 100-fold greater affinity.

The present invention relates to polynucleotide aptamers that specifically bind to OPN and inhibit the binding of osteopontin to its cognate receptors, CD44 and integrin. The sequence of the polynucleotide aptamers of the invention may be selected by any method known in the art. In one embodiment, aptamers may be selected by an iterative selection process such as Systemic Evolution of Ligands by Exponential Enrichment (SELEX). In this type of process, a random pool of oligonucleotides (e.g., about 10⁵ to about 10¹⁵ random oligonucleotides) is exposed to a target protein and the oligonucleotides that bind to the target are isolated and mutagenized and the process repeated until oligonucleotides that bind with the desired affinity to the target are identified. In another embodiment, aptamers may be selected by starting with the sequences and structural requirements of the aptamers disclosed herein and modifying the sequences to produce other aptamers.

In one embodiment of the invention, the aptamers are directed to a mammalian OPN protein (also known as bone sialoprotein I, secreted phosphoprotein I (Spp1), 2ar, uropontin, and early T-lymphocyte activation-1 (Eta-1)). In a further embodiment, the aptamers may be directed to human or mouse OPN. In another embodiment, the aptamers are directed to both human and mouse OPN. The aptamers may bind OPN with a K_{d} of less than about 1000 nM, e.g., less than about 500, 200, 100, 50, or 20 nM. The aptamers may be directed to any isoform of OPN or any combination of isoforms, including one or more of the splice variants OPN-a, OPN-b, and OPN-c (Saitoh et al., Lab. Invest. 72:55 (1995)).

The length of the aptamers of the invention is not limited, but typical aptamers have a length of about 10 to about 100 nucleotides, e.g., about 20 to about 80 nucleotides, about 30 to about 50 nucleotides, or about 40 nucleotides. In certain embodiments, the aptamer may have additional nucleotides attached to the 5′- and/or 3′ end. The additional nucleotides may be, e.g., part of primer sequences, restriction endonuclease sequences, or vector sequences useful for producing the aptamer.

The polynucleotide aptamers of the present invention may be comprised of ribonucleotides only (RNA aptamers), deoxyribonucleotides only (DNA aptamers), or a combination of ribonucleotides and deoxyribonucleotides. The nucleotides may be naturally occurring nucleotides (e.g., ATP, TTP, GTP, CTP, UTP) or modified nucleotides. Modified nucleotides refers to nucleotides comprising bases such as, for example, adenine, guanine, cytosine, thymine, and uracil, xanthine, inosine, and queuosine that have been modified by the replacement or addition of one or more atoms or groups. Some examples of types of modifications that can comprise nucleotides that are modified with respect to the base moieties, include but are not limited to, alkylated, halogenated, thiolated, aminated, amidated, or acetylated bases, in various combinations. More specific examples include 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety (e.g., 2′-fluoro or 2′-O-methyl nucleotides), as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles. The term nucleotide is also meant to include what are known in the art as universal bases. By way of example, universal bases include but are not limited to 3-nitropyrrole, 5-nitroindole, or nebularine. Modified nucleotides include labeled nucleotides such as radioactively, enzymatically, or chromogenically labeled nucleotides).

In one embodiment of the invention, the aptamer is a RNA aptamer and comprises a nucleotide sequence that is identical to any of SEQ ID NOS: 1-14 as shown in Table 1. In another embodiment, the RNA aptamer consists of a nucleotide sequence that is identical to any of SEQ ID NOS: 1-14. In a further embodiment, the RNA aptamer comprises a nucleotide sequence that is at least 70% identical, e.g., at least 75%, 80%, 85%, 90%, 91%, 92%, 93%, 94%, 95%, 96%, 97%, 98%, or 99% identical to any of SEQ ID NOS: 1-14. In another embodiment, the aptamer consists of a nucleotide sequence that is at least 70% identical, e.g., at least 75%, 80%, 85%, 90%, 91%, 92%, 93%, 94%, 95%, 96%, 97%, 98%, or 99% identical to any of SEQ ID NOS: 1-14. In a different embodiment, the aptamer comprises a nucleotide sequence that is identical to a fragment of any of SEQ ID NOS: 1-14 of at least 10 contiguous nucleotides, e.g., at least about 15, 20, 25, 30, or 35 contiguous nucleotides. In a further embodiment, the aptamer comprises a nucleotide sequence that is at least 70% identical, e.g., at least 75%, 80%, 85%, 90%, 91%, 92%, 93%, 94%, 95%; 96%, 97%, 98%, or 99% identical to a fragment of any of SEQ ID NOS: 1-14 of at least contiguous 10 nucleotides, e.g., at least about 15, 20, 25, 30, or 35 contiguous nucleotides. In one embodiment, one or more ribonucleotides in the RNA aptamers described above are substituted by a deoxyribonucleotide. In another embodiment, the fragments and/or analogs of the aptamers of SEQ ID NOS: 1-14 have a substantially similar inhibitory activity as one or more of the aptamers of SEQ ID NOS: 1-14. “Substantially similar,” as used herein, refers to an inhibitory activity on one or more OPN functions that is at least about 20% of the inhibitory activity of one or more of the aptamers of SEQ ID NOS: 1-14.

TABLE 1

| Aptamer | |
|---|---|
| Name | Aptamer Sequence |
| OPN-R3 | CGGCCACAGAAUGAAAAACCUCAUCGAUGUUGCAUAGUUG |
| | (SEQ ID NO: 1) |
| AP1 | UCCCCAGCCCAUAGGAUCAAGCCAAACUCUCAUCCGCGAU |
| | (SEQ ID NO: 2) |
| AP2 | UACAACUCCCGCUGGCCCCAACGCUCUACUCCGAGUAACG |
| | (SEQ ID NO: 3) |
| AP3 | UACCCACCGGCCACGGGAACGAUCAGACGUCCCAUAAU |
| | (SEQ ID NO: 4) |
| AP4 | UCAUUCGCCAAAUGGCGAACCAGCCGGUCGCAGCCAGGAU |
| | (SEQ ID NO: 5) |
| AP5 | CUAACCCCGAGGACAUGUCACGCCGCGCAUAGAGAUUCUC |
| | (SEQ ID NO: 6) |
| AP6 | AAAAUUGUGCGGUUUGCAGAUUAGAAGAGGUCCAUUUGUU |
| | (SEQ ID NO: 7) |
| AP7 | CGGCUCUGAUAUGCUUCCUGGAAGCAGCGUUAUAGCCCAC |
| | (SEQ ID NO: 8) |
| AP8 | AGACGUCAGAACCGGAAUAAACAGACGCUUAACUUUAGAA |
| | (SEQ ID NO: 9) |
| AP9 | ACCCGCCGCAGAAAUUCCGCCCAUCCAGGACGCGGCGCAC |
| | (SEQ ID NO: 10) |
| AP10 | AAUGCCCAUGCAGAAGCCAUCAAUCACAACUCGACCCCAA |
| | (SEQ ID NO: 11) |
| AP11 | GCUUCAUGAAAGGCACGAAACCACCGCGCAUGGGA |
| | (SEQ ID NO: 12) |
| AP12 | CGAGAAAUCGAAUUCCCGCGGCCGCCAUGGCGGCCGGGAG |
| | (SEQ ID NO: 13) |
| AP14 | GGCCGCGGGAAUUCGAUUGGGGGAAUUCUAAUACGACUCA |
| | (SEQ ID NO: 14) |

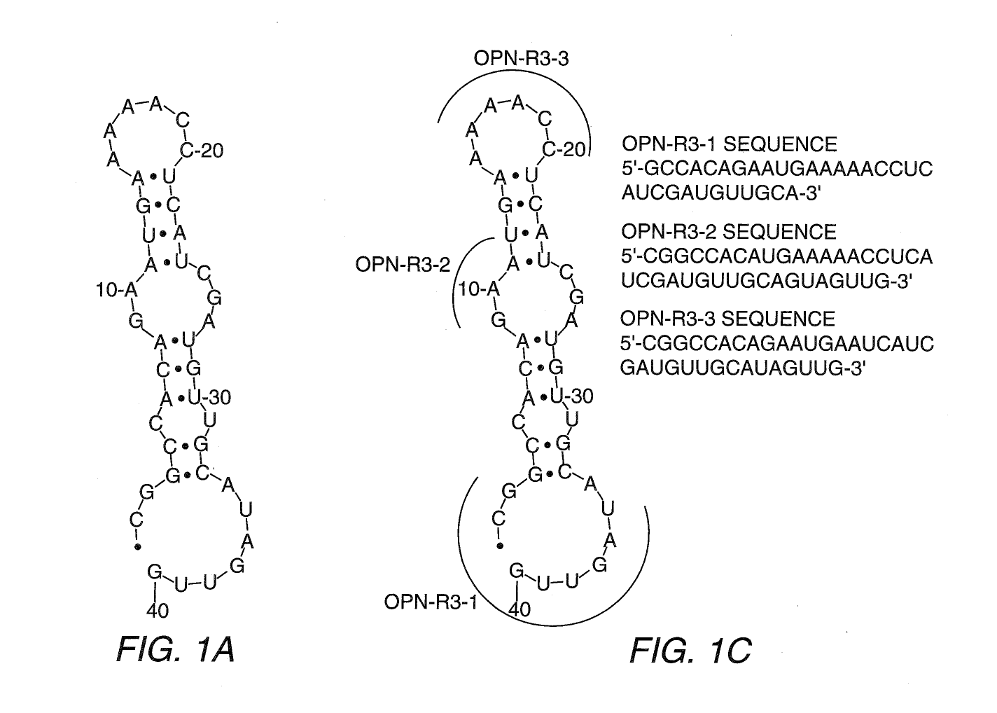

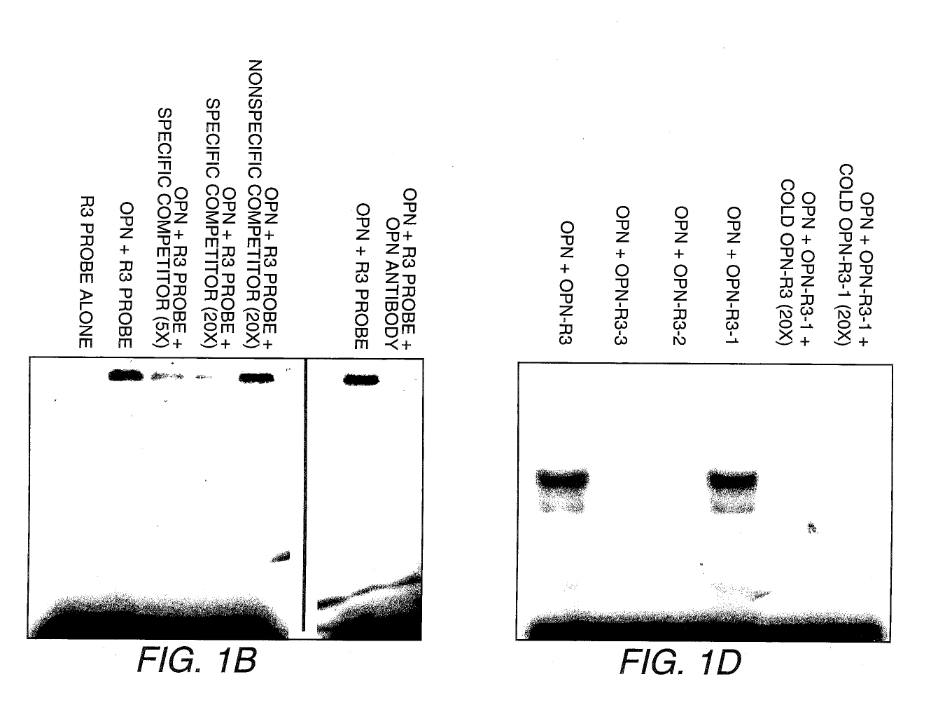

Changes to the aptamer sequences, such as SEQ ID NOS: 1-14, may be made based on structural requirements for binding of the aptamers to OPN. The structural requirements may be readily determined by one of skill in the art by analyzing common sequences between the disclosed aptamers and/or by mutagenizing the disclosed aptamers and measuring OPN binding affinity. For example, each of OPN-R3, APB, AP9, and AP10 comprise the nucleotide sequence CAGAA, suggesting that this sequence is important for binding activity. This importance was confirmed by synthesizing a deletion mutant of OPN-R3 in which nucleotides 9-11 (GAA) were deleted and demonstrating that this mutant did not bind to OPN (FIGS. 1C an 1D, mutant OPN-R3-2). Similarly, deletion of nucleotides 16-20 (AAACC) from OPN-R3 (mutant OPN-R3-3) eliminated OPN binding activity, thereby identifying another structural requirement for binding activity.

Once an aptamer sequence is identified, the aptamer may by synthesized by any method known to those of skill in the art. In one embodiment, aptamers may be produced by chemical synthesis of oligonucleotides and/or ligation of shorter oligonucleotides. Another embodiment of the present invention relates to polynucleotides encoding the aptamers of the invention. The polynucleotides may be used to express the aptamers, e.g., by in vitro transcription, polymerase chain reaction amplification, or cellular expression. The polynucleotide may be DNA and/or RNA and may be single-stranded or double-stranded. In one embodiment, the polynucleotide is a vector which may be used to express the aptamer. The vector may be, e.g., a plasmid vector or a viral vector and may be suited for use in any type of cell, such as mammalian, insect, plant, fungal, or bacterial cells. The vector may comprise one or more regulatory elements necessary for expressing the aptamers, e.g., a promoter, enhancer, transcription control elements, etc. One embodiment of the invention relates to a cell comprising a polynucleotide encoding the aptamers of the invention. In another embodiment, the invention relates to a cell comprising the aptamers of the invention. The cell may be any type of cell, e.g., mammalian, insect, plant, fungal, or bacterial cells.

In one aspect of the invention, the aptamers are modified to increase the circulating half-life of the aptamer after administration to a subject. In one embodiment of the invention, the nucleotides of the aptamers are linked by phosphate linkages. In another embodiment, one or more of the internucleotide linkages are modified linkages, e.g., linkages that are resistant to nuclease degradation. The phrase “modified internucleotide linkage” includes all modified internucleotide linkages known in the art or that come to be known and that, from reading this disclosure, one skilled in the art will conclude is useful in connection with the present invention. Internucleotide linkages may have associated counterions, and the term is meant to include such counterions and any coordination complexes that can form at the internucleotide linkages. Modifications of internucleotide linkages include, without limitation, phosphorothioates, phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that can be saturated or unsaturated and/or substituted and/or contain heteroatoms, linkages with morpholino structures, amides, polyamides wherein the bases can be attached to the aza nitrogens of the backbone directly or indirectly, and combinations of such modified internucleotide linkages. In another embodiment, the aptamers comprise 5′- or 3′-terminal blocking groups to prevent nuclease degradation (e.g., an inverted deoxythymidine or hexylamine).

In a further embodiment, the aptamers are linked to conjugates that increase the circulating half-life, e.g., by decreasing nuclease degradation or renal filtration of the aptamer. Conjugates may include, for example, amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of conjugates also include steroids, such as cholesterol, phospholipids, di- and tri-acylglycerols, fatty acids, hydrocarbons that may or may not contain unsaturation or substitutions, enzyme substrates, biotin, digoxigenin, and polysaccharides. Still other examples include thioethers such as hexyl-S-tritylthiol, thiocholesterol, acyl chains such as dodecandiol or undecyl groups, phospholipids such as di-hexadecyl-rac-glycerol, triethylammonium 1,2-di-O-hexadecyl-rac-glycero-3-H-phosphonate, polyamines, polyethylene glycol, adamantane acetic acid, palmityl moieties, octadecylamine moieties, hexylaminocarbonyl-oxycholesterol, farnesyl, geranyl and geranylgeranyl moieties, such as polyethylene glycol, cholesterol, lipids, or fatty acids. Conjugates can also be detectable labels. For example, conjugates can be fluorophores. Conjugates can include fluorophores such as TAMRA, BODIPY, cyanine derivatives such as Cy3 or Cy5 Dabsyl, or any other suitable fluorophore known in the art. A conjugate may be attached to any position on the terminal nucleotide that is convenient and that does not substantially interfere with the desired activity of the aptamer that bears it, for example the 3′ or 5′ position of a ribosyl sugar. A conjugate substantially interferes with the desired activity of an aptamer if it adversely affects its functionality such that the ability of the aptamer to bind OPN is reduced by greater than 80% in an in vitro binding assay.

A further aspect of the invention relates to pharmaceutical compositions comprising the aptamers of the invention and a pharmaceutically acceptable carrier. In one embodiment, the pharmaceutical compositions comprise a therapeutically effective amount of the aptamers.

Another aspect of the invention relates to methods of using the aptamers of the invention to inhibit the function of OPN. Such methods can be used in vitro and in vivo to study the role of OPN in physiology and disease. The methods may also be used for treatment of cancer and metastases as well as for diagnostic purposes.

One embodiment relates to methods of inhibiting at least one biological function of OPN, comprising contacting OPN with the aptamers of the invention. The biological function may be any biological function known for OPN, including without limitation binding to CD44 or integrin receptors, stimulating the adhesion, migration, or invasion ability of a cell, or stimulating cancer cell metastasis. The inhibition of biological function can be measured by any means known in the art, including the assays described herein.

Another embodiment relates to methods of inhibiting binding of OPN to CD44 and/or integrin receptors, comprising contacting OPN with the aptamers of the invention.

A further embodiment relates to methods of inhibiting the adhesion, migration, invasion ability of a cell, comprising contacting said cell with the aptamers of the invention.

A further embodiment relates to methods of treating diseases and disorders associated with OPN in a subject, comprising administering to said subject the aptamers of the invention. The term “diseases and disorders associated with OPN” refers to any disease or disorder the cause of which or one or more symptoms of which are due at least in part to the presence in a subject of levels of OPN protein at levels higher than the OPN level found in subjects that do not have the disease or disorder. Diseases and disorders associated with OPN include, without limitation, cancer; metastasis; hyperproliferative diseases such as psoriasis; autoimmune diseases such as systemic lupus erythematosus, rheumatoid arthritis, multiple sclerosis, and diabetes; inflammatory diseases such as vasculitis, nephritis, arthritis, osteoarthritis, Crohn's disease, and inflammatory bowel disease; bone diseases such as osteoporosis and osteopetrosis; immune disorders; vascular injuries; restenosis; and atherosclerosis.

Another embodiment relates to methods of treating cancer in a subject, comprising administering to said subject the aptamers of the invention. The methods may be used to treat any type of cancer, e.g., breast, stomach, lung, prostate, liver, or colon cancer.

A further embodiment relates to methods of inhibiting tumor metastasis in a subject, comprising administering to said subject the aptamers of the invention. In one embodiment, the methods are methods of treating or preventing tumor metastasis in a subject. The methods may be used to inhibit metastasis of any type of tumor, e.g., breast, stomach, lung, prostate, liver, or colon cancer tumors.

In the methods of treating cancer or inhibiting tumor metastasis, the aptamers of the invention may be administered to a subject by any suitable route, e.g., intravenously, peritoneally, or intratumorally. In one embodiment, the aptamers are injected regionally, e.g., into blood vessels that lead to a tumor.

Another embodiment relates to methods of promoting wound healing and/or inhibiting scar formation in a subject, comprising administering to said subject the aptamers of the invention. Inhibition of OPN activity at the site of a wound can increase the rate of wound healing as well as decrease the amount of granulation tissue formation and fibrosis that occurs during healing (Mori et al., J. Exp. Med. 205:43 (2008)). In this embodiment, the aptamers may be administered directly to the wound (e.g., topically) and/or systemically.

Inhibition of binding of OPN to its receptors is shown herein to inhibit certain molecular and biochemical pathways and stimulate other pathways. Thus, one aspect of the invention relates to methods of inhibiting and/or stimulating one or more OPN-responsive pathways in vitro (e.g., in cell lines, isolated cells, or isolated tissues) or in a subject using the aptamers of the invention. Inhibition of OPN function is associated with decreased expression of genes associated with several pathways including the interleukin-10 (HO-1 and STAT3 genes), vascular endothelial growth factor (HIF-1A, VEGF), platelet-derived growth factor (PDGF-α, Src), and anti-apoptosis (β-catenin, BCL-2-like protein) pathways. Inhibition of OPN function is also associated with increased expression of genes associated with several pathways including the apoptosis (CAMK2A), granulocyte/macrophage-colony stimulating factor (OSM), anti-proliferative (BTG3-b), and anti-metastasis (CD82) pathways. Thus, one aspect of the invention relates to methods of inhibiting in a subject (e.g., in a cell of the subject) one or more pathways selected from the group consisting of interleukin-10, vascular endothelial growth factor, platelet-derived growth factor, and anti-apoptosis pathways, comprising administering to the subject the polynucleotide aptamers of the invention in an amount effective to inhibit one or more pathways. Another aspect of the invention relates to methods of stimulating in a subject (e.g., in a cell of the subject) one or more pathways selected from the group consisting of apoptosis, granulocyte/macrophage-colony stimulating factor, anti-proliferative, and anti-metastasis pathways, comprising administering to the subject the polynucleotide aptamers of the invention in an amount effective to stimulate one or more pathways.

The decrease in expression of anti-apoptosis associated genes coupled with the enhanced expression of apoptosis inducing genes resulting from inhibition of OPN indicates that inhibition of OPN function may lead to induced apoptosis of cells. Thus, one aspect of the invention relates to methods of inducing apoptosis in a subject (e.g., in a cell of the subject), comprising administering to the subject the polynucleotide aptamers of the invention in an amount effective to induce apoptosis. Further, the inhibition of gene expression in both the VEGF and PDGF pathways resulting from inhibition of OPN indicates that inhibition of OPN function may lead to inhibition of angiogenesis and/or vascularization. Thus, another aspect of the invention relates to methods of inhibiting angiogenesis and/or vascularization in a subject, comprising administering to the subject the polynucleotide aptamers the invention in an amount effective to inhibit angiogenesis and/or vascularization.

For each of the methods described above, the methods may be carried out using a single aptamer targeted to OPN. In another embodiment, the methods may be carried out using two or more different aptamers targeted to OPN, e.g., three, four, five, or six different aptamers.

The aptamers of the present invention may optionally be administered in conjunction with other compounds (e.g., therapeutic agents, chemotherapeutic agents) or treatments (e.g., surgical intervention, angioplasties, radiotherapies) useful in treating diseases and disorders associated with OPN. The other compounds or treatments may optionally be administered concurrently. As used herein, the word “concurrently” means sufficiently close in time to produce a combined effect (that is, concurrently may be simultaneously, or it may be two or more events occurring within a short time period before or after each other). The other compounds may be administered separately from the aptamers of the present invention, or the two combined together in a single composition.

In the case of inflammation, inflammatory diseases, autoimmune disease and other such cytokine mediated disorders, the therapeutic agent(s) may include, without limitation, a nonsteroidal anti-inflammatory drug (NSAID) (such as diclofenac, diflunisal, ibuprofen, naproxen and the like), a cyclooxygenase-2 inhibitor (such as celecoxib, rofecoxib and the like), a corticosteroid (such as prednisone, methylprednisone and the like) or other immunosuppressive agent (such as methotrexate, leflunomide, cyclophosphamide, azathioprine and the like), a disease-modifying antirheumatic drug (DMARD) (such as injectable gold, penicilliamine, hydroxychloroquine, sulfasalazine and the like), a TNF-alpha inhibitor (such as etanercept, infliximab and the like), other cytokine inhibitor (such as soluble cytokine receptor, anti-cytokine antibody and the like), other immune modulating agent (such as cyclosporin, tacrolimus, rapamycin and the like) and a narcotic agent (such as hydrocodone, morphine, codeine, tramadol and the like).

A number of suitable chemotherapeutic agents are contemplated for use in the methods of the present invention. Indeed, the present invention contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics (e.g., gossypol or BH3 mimetics); agents that bind (e.g., oligomerize or complex) with a Bcl-2 family protein such as Bax; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of chemotherapeutic compounds and anticancer therapies suitable for co-administration with the disclosed compounds are known to those skilled in the art.

In further embodiments, chemotherapeutic agents suitable for use in the methods of the present invention include, but are not limited to: 1) vinca alkaloids (e.g., vinblastine, vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

In the case of would healing and scar formation, the therapeutic agent(s) may include, without limitation, angiogenesis promoters (e.g., platelet derived growth factor, vascular endothelial growth factor), anti-inflammatory agents, antiseptic agents (e.g., oxygen- and halogen-releasing compounds); metal compounds (e.g., silver and mercury compounds); organic disinfectants (e.g., formaldehyde-releasing compounds, alcohols, phenols including alkyl- and arylphenols as well as halogenated phenols, quinolines and acridines, hexahydropyrimidines, quaternary ammonium compounds and iminium salts, and guanidines, dithranol), agents promoting granulation and epithelialization (e.g., dexpanthenol, allantoines, azulenes, tannines, and vitamin B-type compounds), proepithelin, secretory leukocyte protease inhibitor, immunosuppressants (e.g., cyclosporine), antimetabolites (e.g., methotrexate), corticosteroids, vitamin D and vitamin D analogs, vitamin A or its analogs (e.g., etretinate), tar, coal tar, anti pruritic and keratoplastic agents (e.g., cade oil), keratolytic agents (e.g., salicylic acid), emollients, lubricants, photosensitizers (e.g., psoralen, methoxsalen), antimicrobial agents, antifungal agents, and antibiotics.

One aspect of the present invention relates to the use of the aptamers of the invention for diagnostic purposes. The aptamers can be used as binding agents in assays for measuring the level of OPN in a subject. Such measurements can be used to determine if OPN levels are abnormal. Such measurements can further be used to diagnose a disease or disorder associated with OPN, e.g., associated with OPN overexpression or underexpression. In other embodiments, the aptamers can be used in OPN receptor competitive binding assays to measure the abundance of OPN receptors and/or the binding affinity and specificity of OPN for the receptors. The aptamers can also be used for in vivo imaging or histological analysis. Numerous suitable binding assays are well known to those of skill in the art. Diagnostic assays can be carried out in vitro on isolated cells or cell lines for research purposes. Diagnostic assays can also be carried out on samples from a subject (e.g., tissue samples (biopsies, aspirates, scrapings, etc.) or body fluid samples (blood, serum, saliva, urine, cerebrospinal fluid, etc.)) or carried out in vivo. The aptamers can be labeled using methods and labels known in the art including, but not limited to, fluorescent, luminescent, phosphorescent, radioactive, and/or colorimetric compounds.

In one aspect, the invention relates to a method of measuring the level of OPN in a subject, comprising the step of using the polynucleotide aptamer of the invention to bind OPN. In another aspect, the invention relates to a method of diagnosing a disease or disorder associated with OPN in a subject, comprising the step of measuring the level of OPN in the subject using the polynucleotide aptamer of the invention. The level of OPN can then be correlated with the presence or absence of a disease or disorder associated with OPN.

The present invention is primarily concerned with the treatment and diagnosis of human subjects, but the invention may also be carried out on animal subjects, particularly mammalian subjects such as mice, rats, dogs, cats, livestock and horses for veterinary purposes, and for drug screening and drug development purposes.

### Pharmaceutical Formulations.

The aptamers described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the aptamer is typically admixed with, inter alia, an acceptable carrier. The carrier must, of course, be acceptable in the sense of being compatible with any other ingredients in the formulation and must not be deleterious to the patient. The carrier may be a solid or a liquid, or both, and may formulated with the aptamer as a unit-dose formulation, for example, a tablet, which may contain from 0.01 or 0.5% to 95% or 99% by weight of the aptamer. One or more aptamers may be incorporated in the formulations of the invention, which may be prepared by any of the well known techniques of pharmacy comprising admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the aptamer and a suitable carrier (which may contain one or more accessory ingredients as noted above). In general, the formulations of the invention are prepared by uniformly and intimately admixing the aptamer with a liquid or finely divided solid carrier, or both, and then, if necessary, shaping the resulting mixture. For example, a tablet may be prepared by compressing or molding a powder or granules containing the aptamer, optionally with one or more accessory ingredients. Compressed tablets may be prepared by compressing, in a suitable machine, the aptamer in a free-flowing form, such as a powder or granules optionally mixed with a binder, lubricant, inert diluent, and/or surface active/dispersing agent(s). Molded tablets may be made by molding, in a suitable machine, the powdered aptamer moistened with an inert liquid binder.

Formulations suitable for buccal (sub-lingual) administration include lozenges comprising the aptamer in a flavored base, usually sucrose and acacia or tragacanth; and pastilles comprising the aptamer in an inert base such as gelatin and glycerin or sucrose and acacia.

Formulations of the present invention suitable for parenteral administration comprise sterile aqueous and non-aqueous injection solutions of the aptamer, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising one or more aptamers, in a unit dosage form in a sealed container. The aptamer is provided in the form of a lyophilizate which is capable of being reconstituted with a suitable pharmaceutically acceptable carrier to form a liquid composition suitable for injection thereof into a subject. The unit dosage form typically comprises from about 1 mg to about 10 grams of the compound. When the aptamer is substantially water-insoluble (e.g., when conjugated to a lipid), a sufficient amount of emulsifying agent which is physiologically acceptable may be employed in sufficient quantity to emulsify the aptamer in an aqueous carrier. One such useful emulsifying agent is phosphatidyl choline.

Formulations suitable for rectal administration are preferably presented as unit dose suppositories. These may be prepared by admixing the aptamer with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharm. Res. 3:318 (1986)) and typically take the form of an optionally buffered aqueous solution of the aptamer. Suitable formulations comprise citrate or bis\tris buffer (pH 6) or ethanol/water and contain from 0.1 to 0.2 M active ingredient.

Further, the present invention provides liposomal formulations of the aptamers disclosed herein. The technology for foaming liposomal suspensions is well known in the art. When the aptamer is in the form of an aqueous-soluble material, using conventional liposome technology, the same may be incorporated into lipid vesicles. In such an instance, due to the water solubility of the aptamer, the aptamer will be substantially entrained within the hydrophilic center or core of the liposomes. The lipid layer employed may be of any conventional composition and may either contain cholesterol or may be cholesterol-free. When the aptamer of interest is water-insoluble, again employing conventional liposome formation technology, the aptamer may be substantially entrained within the hydrophobic lipid bilayer which forms the structure of the liposome. In either instance, the liposomes which are produced may be reduced in size, as through the use of standard sonication and homogenization techniques.

Of course, the liposomal formulations containing the aptamer disclosed herein, may be lyophilized to produce a lyophilizate which may be reconstituted with a pharmaceutically acceptable carrier, such as water, to regenerate a liposomal suspension.

Other pharmaceutical compositions may be prepared from the aptamer disclosed herein, such as aqueous base emulsions. In such an instance, the composition will contain a sufficient amount of pharmaceutically acceptable emulsifying agent to emulsify the desired amount of the aptamer. Particularly useful emulsifying agents include phosphatidyl cholines and lecithin.

In addition to aptamer, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

### Dosage and Routes of Administration.

As noted above, the present invention provides pharmaceutical formulations comprising the aptamers of the invention, in pharmaceutically acceptable carriers for oral, rectal, topical, buccal, parenteral, intramuscular, intradermal, or intravenous, and transdermal administration.

The therapeutically effective dosage of any one active agent, the use of which is in the scope of present invention, will vary somewhat from compound to compound, and patient to patient, and will depend upon factors such as the age and condition of the patient and the route of delivery. Such dosages can be determined in accordance with routine pharmacological procedures known to those skilled in the art. As a general proposition, a dosage from about 0.001 or 0.01 to about 250 or 500 mg/kg will have therapeutic efficacy, with all weights being calculated based upon the weight of the active compound, including the cases where a salt is employed. Toxicity concerns at the higher level may restrict intravenous dosages to a lower level such as up to about 10 mg/kg, with all weights being calculated based upon the weight of the active base, including the cases where a salt is employed. A dosage from about 1 mg/kg to about 200 mg/kg may be employed for oral administration. Typically, a dosage from about 0.1 mg/kg to 100 mg/kg may be employed for intramuscular injection. The duration of the treatment is usually once per day for a period of two to three weeks or until the condition is essentially controlled. The treatment may be administered more frequently than once per day (e.g., 2, 3, or 4 times per day) or less frequently than once per day (e.g., once every 2, 3, 4, 5, or 6 days or once every 1, 2, 3, or 4 weeks). Lower doses given less frequently can be used prophylactically to prevent or reduce the incidence of recurrence of the disease.

The present invention is explained in greater detail in the following non-limiting Examples.

### EXAMPLE 1

### Development and Characterization of RNA Aptamers Directed Against Osteopontin

RNA aptamers to OPN were prepared using the Systematic Evolution of Ligands by Exponential Enrichment (SELEX) technique. SELEX is an iterative in vitro selection process consisting of sequential selection and amplification steps that can efficiently reduce a complex library of nucleic acids with randomized sequences (complexity of approx. 10¹⁴) to a minimized subset of one or more sequences that bind tightly to the target of choice. A random pool of RNA oligonucleotides was generated by in vitro transcription of synthetic DNA templates following the instructions in the DuraScribe T7 Transcription Kit (Epicentre Biotech, Madison, Wis.). 2-Fluorine-dCTP, 2-Fluorine-dUTP, normal GTP and ATP are efficiently incorporated into RNA transcripts through the DuraScribe T7 RNA polymerase. SELEX was applied by alternating the bait protein between human OPN and mouse OPN in order to achieve RNA aptamer targeting to common features of both proteins. Selection after Round 8 through 11 was followed by ligation of 0.5 μg of the double stranded DNA pool into PGEM-T vector systems (Promega, Madison, Wis.) for sequencing. The DNA sequence used for in vitro transcription was

(SEQ ID NO: 15)
5′-GGGGGAATTCTAATACGACTCACTATAGGGAGGACGATGCGG-
N40-CAGACGACTCGCTGAGGATCCGAGA-3′,

where N40 represents the 40 nucleotide RNA aptamer library sequence. Following successive rounds of SELEX, 14 OPN aptamers (SEQ ID NOS: 1-14) were identified. Of these 14, an aptamer termed OPN-R3 (SEQ ID NO: 1) was selected. The sequence of OPN-R3 and a mutant version of OPN-R3 follows, where C denotes 2-Fluorine-dCTP and U denotes 2-Fluorine-dUTP.

OPN-R3:
(SEQ ID NO: 1)
5′-CGGCCACAGAAUGAAAAACCUCAUCGAUGUUGCAUAGUUG-3′
Mutant OPN-R3:
(SEQ ID NO: 16)
5′-CGGCCACAGAAU [symbol] CAUCGAUGUUGCAUAGUUG-3′

The RNA-protein equilibrium dissociation constant (K_{d}) of OPN-R3 was characterized using the double-filter nitrocellulose filter binding method (Gopinath, Anal. Bioanal. Chem. 387:171 (2007)). For all binding assays, RNAs were dephosphorylated using bacterial alkaline phosphatase (Invitrogen, Carlsbad, Calif.) and 5′-end labeled using T4 polynucleotide kinase (New England Biolabs, Beverly, Mass.) and γ-³²P-ATP (MP Biomedicals, Solon, Ohio). Direct binding assays were carried out by incubating ³²P-labeled RNA at a concentration of less than 0.1 nM and target protein at concentrations ranging from 300 nM to 10 pM in selection buffer at 37° C. The fraction of RNA bound was quantified with a Phosphorlmager (Molecular Dynamics, Sunnyvale, Calif.). Raw binding data were corrected for nonspecific background binding of radiolabeled RNA to the nitrocellulose filter. Following the eighth round of SELEX, OPN-R3 was found to have a K_{d} of 18±0.2 nM. The predicted secondary structure of OPN-R3 contains the usual stem-loop structure of RNA aptamers and is shown in FIG. 1A.

To confirm in vitro binding of OPN-R3 to OPN, RNA electrophoretic mobility shift assays (REMSA) were performed (FIG. 1B). REMSA were conducted in freshly prepared buffers containing protease inhibitors and dithiothreitol (1 mM). Recombinant human OPN (100 nM) was dissolved in ice-cold buffer C containing 20 mM HEPES, pH 7.9, 0.4 M NaCl, 1.0 mM EDTA, 1.0 mM EGTA, 1.0 mM dithiothreitol, pepstatin A (2 μg/ml), and 0.5 mM phenylmethylsulfonyl fluoride, aliquoted and stored at −80 ° C. until use. OPN-R3 and mutant OPN-R3 RNA aptamers were synthesized, and then end-labeled with [γ-³²P] ATP (2500 Ci/mmol) using T4 polynucleotide kinase (Promega), followed by G-50 column purification. The reactions were resolved on 6% native acrylamide gel in 0.5× Tris-borate/EDTA buffer and visualized by autoradiography. In specific competitive binding assays, unlabeled OPN-R3 type aptamers were added at a 20-fold molar excess. In nonspecific competitive binding assays, unlabeled mutant OPN-R3 aptamers were used. Supershift assays were performed by preincubating recombinant human OPN with rabbit anti-human OPN polyclonal Ab (Santa Cruz Biotechnology, Santa Cruz, Calif.).

Human OPN bound to OPN-R3; increasing concentrations of unlabelled OPN-R3 probe effectively competed for OPN binding, while unlabelled nonspecific competitor RNA did not alter OPN binding to OPN-R3. Supershift assays using antibody to human OPN demonstrated decreased binding of OPN to OPN-R3. These data indicate that human OPN binds to OPN-R3 in a specific fashion.

Mutagenesis of OPN-R3 was then performed to determine the active binding site. FIG. 1C depicts the regions of OPN-R3 that underwent deletion. Deletion constructs, OPN-R3-1, OPN-R3-2 and OPN-R3-3, were then tested in REMSA with human OPN (FIG. 1D). Only OPN-R3-1 retained its OPN binding abilities, suggesting that regions 2 and 3 are both required for in vitro binding to OPN.

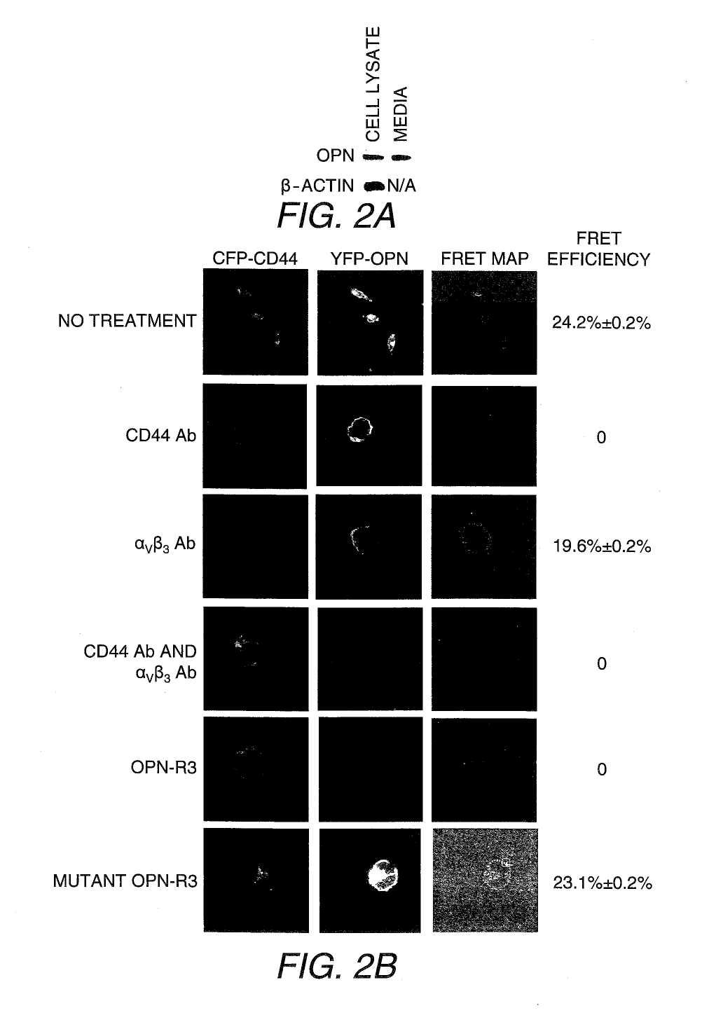

The efficacy of OPN-R3 for inhibition of OPN binding to its cell surface receptors, CD44 and αᵥβ₃ integrin, in MDA human breast cancer cells was determined. The MDA-MB-231 human breast cancer cell line was obtained from the American Type Culture Collection (Manassas, Va.) and cultured in Dulbecco's modified Eagle's medium (DMEM) supplemented with 10% fetal calf serum, penicillin (100 units/ml), and streptomycin (100 μg/ml), and maintained at 37° C. in a humidified atmosphere of 5% CO₂. Western blot analysis confirmed production of OPN in MDA cell lysates and media (FIG. 2A). Cells were lysed in buffer (0.8% NaCl, 0.02% KCl, 1% SDS, 10% Triton X-100, 0.5% sodium deoxycholic acid, 0.144% Na₂HPO₄, and 0.024% KH₂PO₄, 2 mM phenylmethylsulfonyl fluoride, pH 7.4) and centrifuged at 12,000×g for 10 min at 4° C. The protein concentration was determined by the Bio-Rad protein assay kit; the protein samples were separated by 4-20% SDS-PAGE and electrotransferred onto polyvinylidene difluoride membranes (Amersham Biosciences) by semi-dry transfer (Bio-Rad, Calif.). The membranes were probed with the primary antibody for 1 h at room temperature. The antibody was then detected using the appropriate horseradish peroxidase-conjugated secondary antibody. The reactive proteins were visualized by means of the ECL kit (Amersham Bioscience).

FRET confocal microscopy was used to detect OPN binding to the CD44 receptor. Human full length CD44s cDNA (a gift from Dr. David Waugh, Queen's University Belfast, UK) and human OPN cDNA were separately fused in frame into mammalian expression vector pECFP and pEYFP (BD Biosciences Clontech, Mountain View, Calif.), respectively. MDA-MB231 cells were cultured on coverslips and then co-transfected with pECFP-CD44 FRET donor and pEYFP-OPN FRET acceptor plasmids. Cells were transiently transfected using Lipofectamine 2000 according to manufacturer's instruction (Invitrogen, MD). Briefly, 4×10⁵ cells were seeded with antibiotic-free DMEM medium on each well of 12-well plates the day before transfection. Two micrograms of plasmid DNA and 4 μl Lipofectamine 2000, diluted with Opti-MEM medium, were mixed gently and incubated with cells. Culture medium was changed after 6 h transfection and incubated further at 37° C. for 24 h. The control cells received Lipofectamine 2000 alone. After 24 h post transfection, the coverslips were rinsed three times with ice-cold PBS followed by fixation for 15 min with 1% (w/v) paraformaldehyde. Coverslips were rinsed three times with PBS and mounted onto a microscope slide using 50 μl mounting medium (Calbiochem, Germany). The coverslips were sealed by wax and kept at 4° C. until analysis. Leica TCS SP2 confocal microscope was used for image acquisition. CFP and YFP emission spectra were first optimized at 458 nm and 514 nm, respectively. FRET was measured by acceptor photobleaching using the FRET-AB wizard in the Leica TS software. A pair of pre-bleach images of CFP and YFP images were collected for the cells of interest. Randomly chosen regions of interest were irradiated (bleached) with the 514-nm laser line set at 100% intensity to photobleach YFP only for the minimum number of iterations of bleaching required. Post-bleach CFP and YFP images were collected following photobleaching. FRET was indicated by an increase in CFP donor fluorescence intensity following YFP photobleaching. FRET efficiency was calculated as 100×[(Donor post-bleach−Donor prebleach)/Donor post-bleach], taking into account CFP and YFP background noise in each channel; FRET efficiency was measured and calculated automatically by Leica LAS AF software.

Confocal fluorescence microscopy showed overlapping localization of both CFP-tagged CD44 and YFP-tagged OPN to the MDA plasma membrane (FIG. 2B). Acceptor photobleaching was then used to measure FRET between CFP-CD44 and YFP-OPN. If CFP-CD44 (donor) and YFP-OPN (acceptor) are within 10 nm of each other, and the fluorophore dipoles are aligned, resonance energy can be transferred from CFP to YFP (Mi et al., Cancer Res. 67:4088 (2007)). To perform acceptor photobleaching, a pre-bleach image was captured using the 458-nm laser line; a region of the plasma membrane was selectively irradiated using the 514-nm laser line. An increase in CFP fluorescence was observed following YFP photobleaching, and the mean FRET efficiency was 24.2±0.2%. Acceptor photobleaching experiments were done on 50 MDA-MB231 cells (three regions per cell) coexpressing CFP-CD44 and YFP-OPN as well as on a similar number of cells in which no FRET was expected. These additional controls included (a) cotransfection of CFP- and YFP-empty plasmids (FRET efficiency, 0.23±0.2%) and (b) transfection of a CFP-empty plasmid alone (FRET efficiency, 0.21±0.1%). These data confirm interaction between OPN and CD44 on the MDA-MB231 cell surface.

Subsequent FRET experiments were then performed in the presence of OPN-R3, blocking antibody to CD44, and/or blocking antibody to αᵥβ₃ integrin. In the presence of CD44 antibody, cell surface binding of OPN was still present but no FRET was detected, indicating OPN binding to alternative αᵥβ₃ integrin binding sites (FIG. 2B). In the presence of blocking antibody to αᵥβ₃ integrin, FRET was detected (19.6±0.2%; p=NS vs. CD44 antibody) and cell surface OPN was present, suggesting that OPN was bound to native CD44 and/or CFP-CD44 receptors. However, in the presence of both CD44 antibody and αᵥβ₃ integrin antibody, neither cell surface OPN nor FRET was detected. Finally, in the presence of OPN-R3 (100 nM), FRET was totally ablated, and no cell surface OPN was found, suggesting that the RNA aptamer blocked all interaction of OPN with its cell surface receptors, including CD44. As a control, mutated OPN-R3 aptamer was associated with FRET of 23.1±0.2%.

### EXAMPLE 2

### OPN-R3 and OPN-Dependent Signaling Pathways

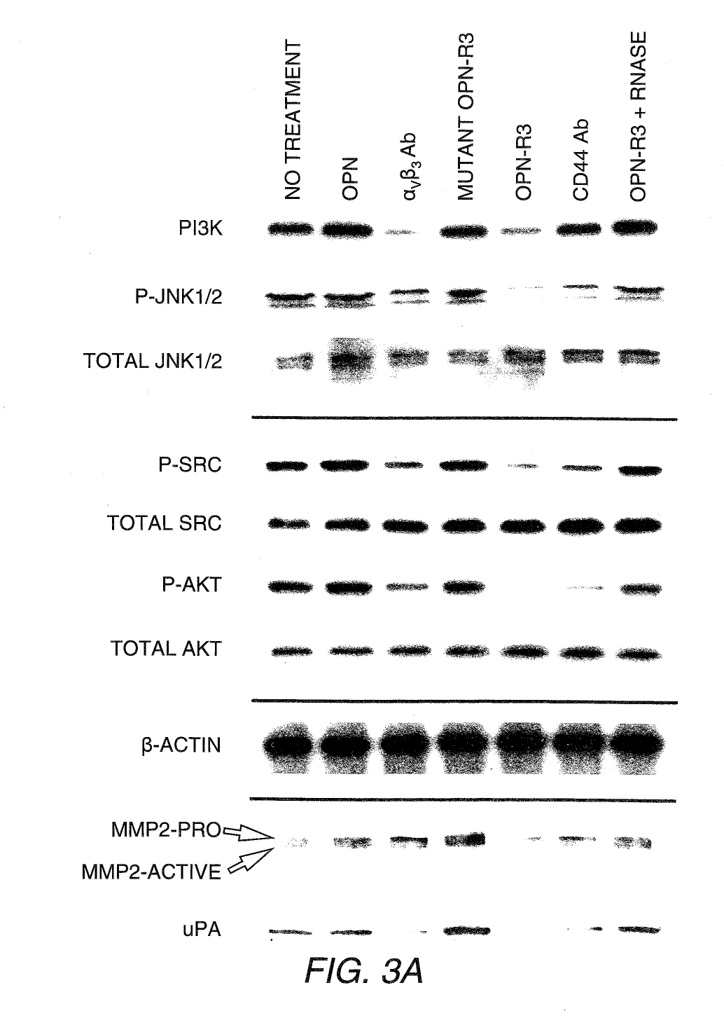

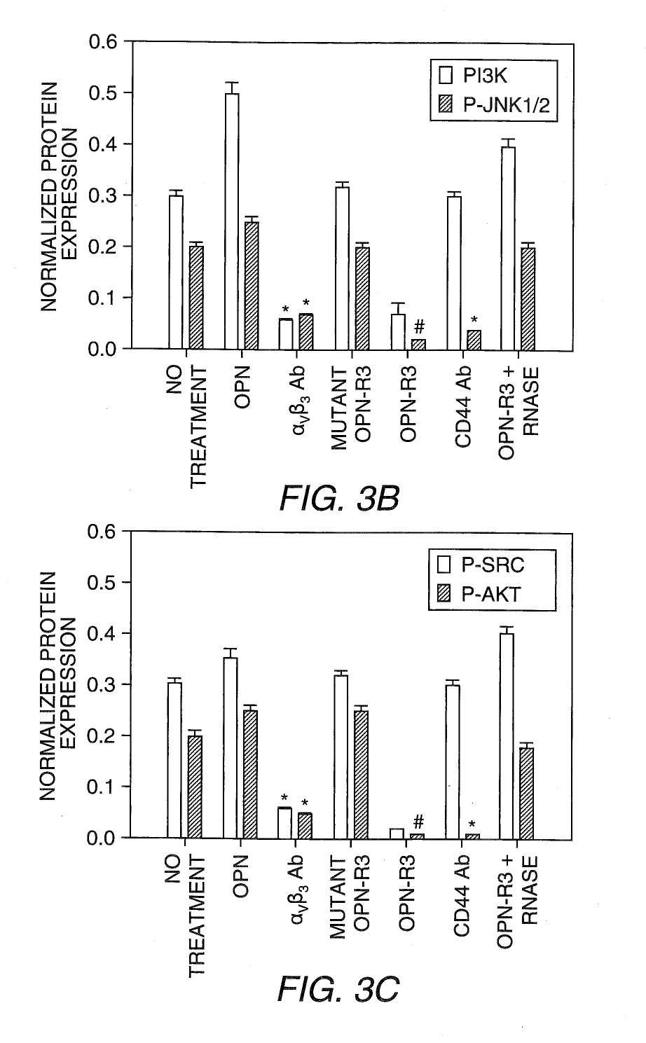

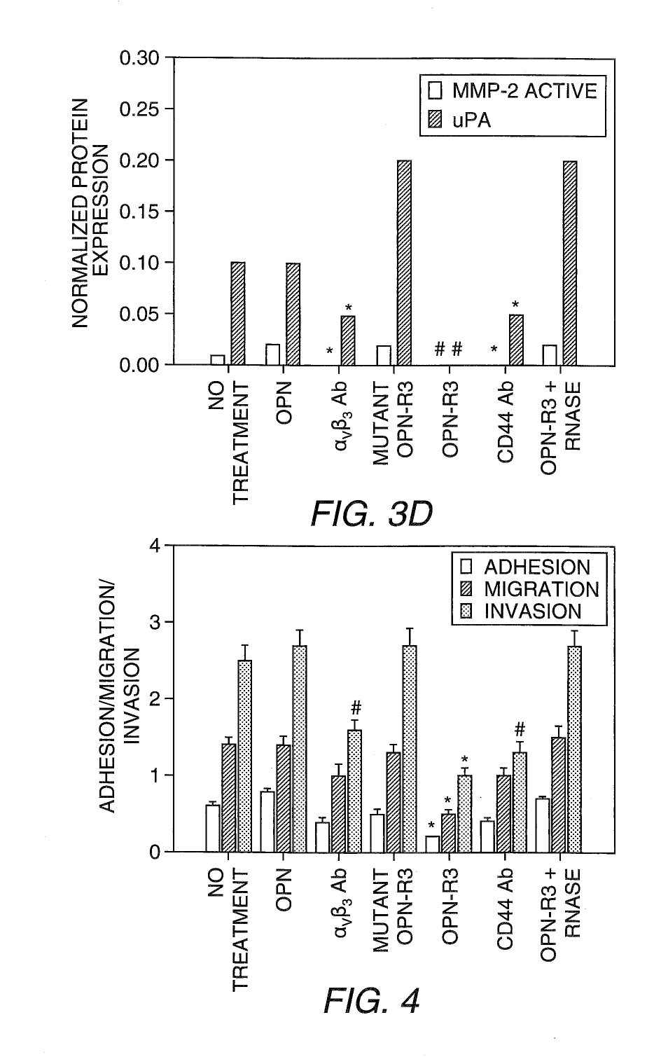

To determine the effect of OPN-R3 on OPN dependent signal pathways, Western blots were performed in MDA-MB231 cells as described above for Src, P-Src (Cell Signaling, Beverly, Mass.), PI3K, JNK, P-JNK, Akt, and P-Akt as constituents of the αᵥβ₃ and/or CD44 pathways. The expression of these various markers was assessed in response to exposure to OPN-R3, exogenous OPN (20 nM), αᵥβ₃ antibody, CD44 antibody, mutant OPN-R3 and/or mutant OPN-R3 with RNase (FIG. 3). Expression of phosphorylated JNK-1/2 (P-JNK1/2) and PI3K was detected in untreated MDA cells and was not altered in the presence of exogenous OPN. Exposure of the cells to αᵥβ₃ Ab or OPN-R3 significantly decreased both P-JNK1/2 and PI3K expression. In contrast, mutant OPN-R3 and OPN-R3+RNase did not alter levels of P-JNK1/2 and PI3K. Interestingly, exposure of the MDA cells to CD44 antibody did not alter PI3K, but did decrease P-JNK1/2, suggesting that crosstalk or overlap might exist between the CD44 and αᵥβ₃ integrin signal transduction pathways. When phosphorylated-Src (P-Src) and -Akt (P-Akt) were addressed, expression of both proteins was detected in untreated MDA cells and was not altered in the presence of exogenous OPN (20 nM). Exposure of the cells to CD44 antibody or OPN-R3 significantly decreased P-Src and P-Akt expression. In contrast, mutant OPN-R3 and OPN-R3+RNase had no discernable effect. Antibody to αᵥβ₃ integrin decreased PI3K and P-Src expression also; this repeats the theme of overlapping signal transduction pathways between CD44 and αᵥβ₃ integrin receptors.

OPN has previously been demonstrated to partially regulate expression of matrix metalloproteinase 2 (MMP2) and uroplasminogen activator (uPA) as mediators of extracellular matrix degradation and facilitators of metastasis. In this setting, expression of pro- and active-MMP2 and uPA in MDA-MB231 cells was examined following exposure to OPN-R3 (FIG. 3D). In a fashion similar to that seen for the previous proteins, pro-MMP2, active MMP2 and uPA were detected in untreated MDA-MB231 cells. Exogenous OPN did not significantly alter expression. OPN-R3 ablated uPA and active MMP2 levels, although pro-MMP2 was still readily detected. Antibody to CD44 and αᵥβ₃ integrin significantly decreased uPA, pro-MMP2 and active MMP2 levels. Mutant OPN-R3 and OPN-R3+RNase had no effect. In total, these data indicate that OPN-R3 aptamer can significantly decrease activation and/or expression of various constituents of the CD44 and αᵥβ₃ integrin signal transduction pathways and their downstream effector molecules in MDA-MB231 cells.

### EXAMPLE 3

### OPN-R3 and MDA-MB231 Adhesion, Migration, and Invasion

To assess the functional consequences of OPN-R3 ligation of OPN, in vitro adhesion, migration and invasion assays were performed. Adhesion assays was performed on 96-well microtiter plates coated with 10 μg/ml Matrigel. Cells were trypsinized and resuspended in DMEM with 1% BSA, 1 mM MgCl₂, 0.5 mM CaCl₂ at a concentration of 1×10⁶ cells/ml. 1×10⁵ cells (100 μl) were added into each well and placed for 30 min at 37° C. in 5% CO₂ humidified air incubation. Non-adherent cells were removed by gently washing the wells three times with phosphate-buffered saline (PBS) with 1 mM MgCl₂ and 0.5 mM CaCl₂. Adherent cells were fixed with 3.7% paraformaldehyde for 10 min at room temperature, followed by rinsing with PBS, and stained with 0.4% crystal violet for 10 min. After extensive rinsing, the dye was released from the cells by addition of 30% acetic acid, and the microtiter plates were read in a microplate reader (Molecular Devices, Berkeley, Calif.) at 590 nm.

The migration and invasion assay were carried out in a Boyden Chamber system (Corning, N.Y.). Cells were seeded at a density of 10⁵ cells per well in triplicate in the upper chamber of 12 well transwells (8 μm pore). After being incubated at 37° C. with 5% CO₂ for 24 hours, the cells were fixed in 3.7% paraformaldehyde in phosphate-buffered saline for 10 min. The cells on the top surface of the filters were wiped off with cotton swabs. Following three washes with PBS, the filters were stained with 0.4% crystal violet for 10 min, and the dye was detected as described for the in vitro adhesion assay.

When compared to untreated cells, adhesion, migration and invasion in OPN-R3 treated cells were decreased by 60%, 50%, and 65%, respectively (FIG. 4). In comparison, αᵥβ₃ integrin antibody decreased adhesion, migration and invasion by 30%, 40%, and 45%, respectively. Similarly, CD44 antibody decreased adhesion, migration and invasion by 40%, 30%, and 48%, respectively. Exogenous OPN, mutant OPN-R3 and OPN-R3+RNase had no effect on the three measures. These results indicate that OPN-R3 can effectively and significantly inhibit the in vitro correlates of adhesion, migration and invasion in MDA-MB231 cells.

### EXAMPLE 4

### Functional In Vivo Activity of OPN-R3

In the following in vivo studies, OPN-R3 (and mutant OPN-R3-2) was modified to increase its biological half-life, incorporating 2′-O-methyl substituted nucleotides, 5′-cholesterol modification and 3′-inverted deoxythymidine. The sequence of OPN-R3 aptamer used in the in vivo studies is the same as OPN-R3-1. The half-life of the modified RNA aptamer oligo was >24 hours in human serum at 37° C. The half-life of both OPN-R3 and mutant OPN-R3-2 in Dulbecco's modified Eagle's medium with 10% normal mouse serum was 8 hours. The in vitro K_{d} of the modified OPN-R3 was 18 nmol/l; in vitro specific binding of modified OPN-R3 to OPN was again confirmed using REMSA.

A xenograft model of MDA-MB231 cell implantation into the mammary fat pads of female NOD scid mice was used. The MDA cells were previous engineered to express luciferase. MDA-MB231 cells (1×10⁶) were suspended in 50% Matrigel-Hanks balanced salt solution and implanted into the R4 or L4 positions of the mammary fat pad of 6-week-old female NOD scid mice (four per group). Vehicle, modified OPN-R3, or mutant OPN-R3 (500 μg/kg each) were injected into the mouse tail vein every 2 days. Mice were anesthetized with intraperitoneal ketamine (75 mg/kg) and xylazine (10 mg/kg). For bioluminescent imaging, animals were placed in a light-tight chamber in which grayscale reference images were obtained under dim conditions. A pseudocolor image acquired in the dark was superimposed on the grayscale image to represent photons emitted from tumors. Bioluminescence is reported as the sum of detected photons per second from a constant region of interest. Ten minutes after administration of luciferase substrates (D-luciferin, 150 mg/kg), anesthetized mice were imaged with the IVIS 100 Imaging System (Xenogen, Alameda, Calif.) following the company's manual. Initial in vivo images at day 2 were obtained to establish baseline tumor volume as measured by photon emission.

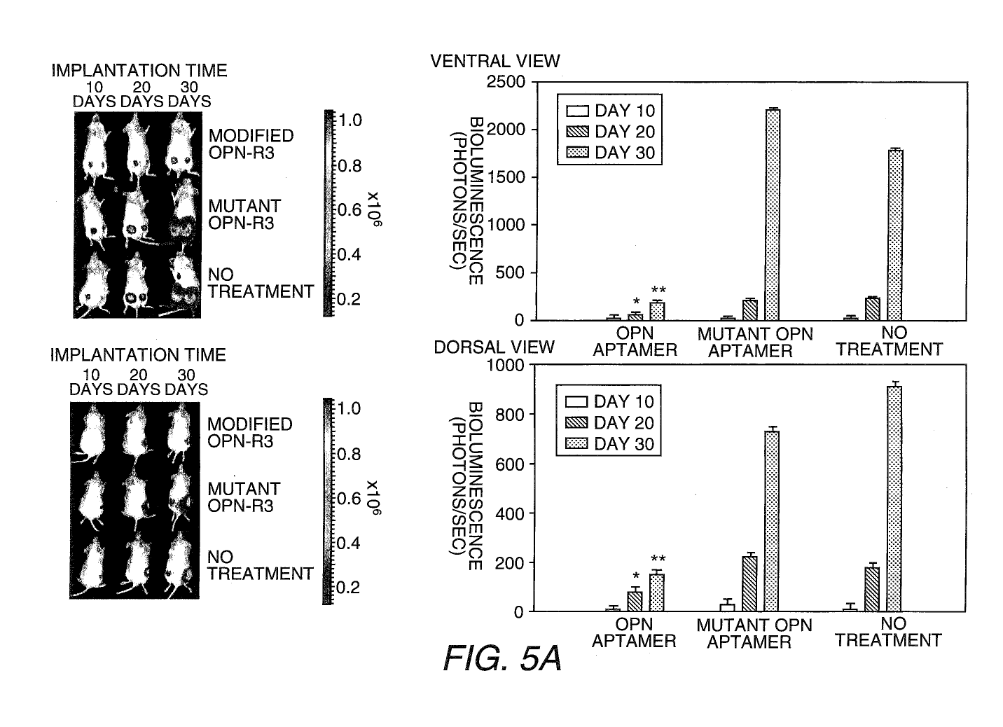

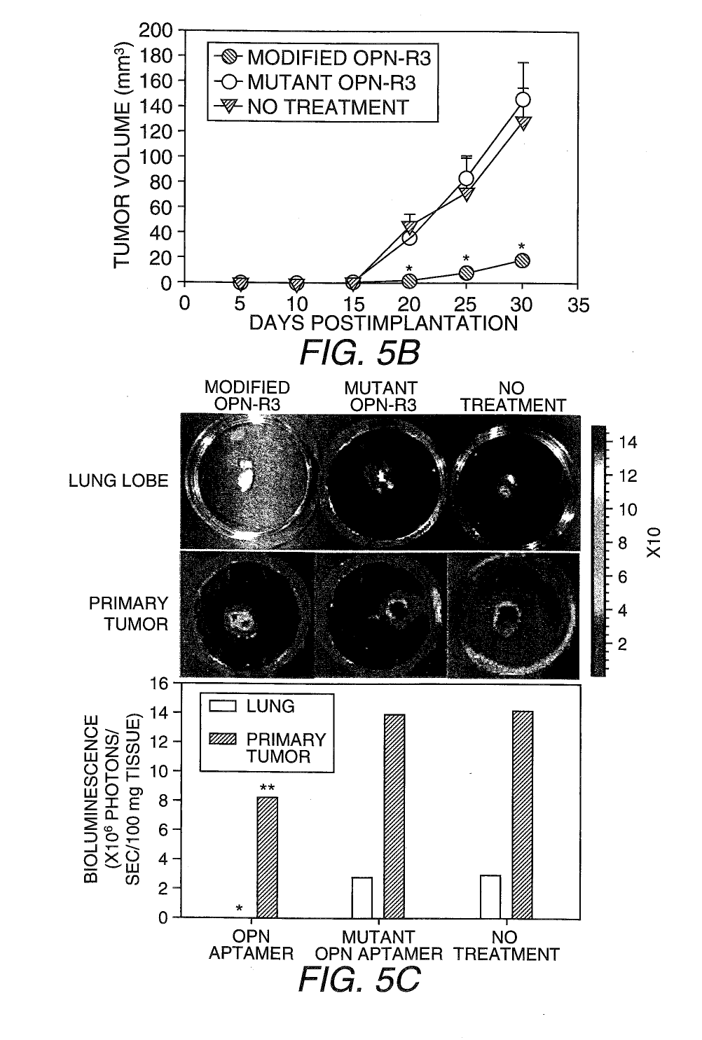

Bioluminescence imaging data at days 10, 20, and 30 are displayed in FIG. 5. Bioluminescence was significantly decreased in the modified OPN-R3-treated animals by over 4- and 12-fold at 20 and 30 days after implantation, respectively, when compared to mutant OPN-R3 or vehicle-treated animals (P<0.01 at 20 days and 30 days for OPN-R3 vs. mutant OPN-R3 and vehicle). Tumor volumes were measured on a daily basis (FIG. 5B). Similar to that seen with the bioluminescence data, tumor volume was significantly deceased in the modified OPN-R3-treated animals. At day 20, tumor volume in the modified OPN-R3 aptamer-treated group was 18-20-fold smaller than that noted in the mutant OPN-R3 and vehicle groups (P<0.01 vs. mutant OPN-R3 and vehicle). At day 30, modified OPN-R3 aptamer-treated group tumor size was eight-fold less than that of the mutant OPN-R3 and the vehicle groups (P<0.01 vs. mutant OPN-R3 and vehicle).

For ex vivo imaging, after eight weeks of modified OPN-R3 or mutant OPN-R3 treatment, D-luciferin (150 mg/kg) was injected into the mice before necropsy. Lung lobes were excised, weighed, placed into tissue culture plates with D-luciferin (300 μg/ml) in PBS, and imaged. The mean bioluminescence was quantified and analyzed using Living Image software (Xenogen). Bioluminescence from ROI was defined manually. At 8 weeks, necropsy tissue from lung and primary tumor locations were examined for bioluminescence in a site for potential metastases and in the primary location (FIG. 5C). In lung tissue, the measured bioluminescence in the modified OPN-R3 group was <1% of that noted in the mutant OPN-R3 and vehicle groups (P<0.01 vs. mutant OPN-R3 and vehicle). These data indicate that modified OPN-R3 aptamer can significantly decrease both local tumor growth and distant metastases of MDA-MB231 cells in this xenograft model.

### EXAMPLE 5

### Gene Expression Analysis

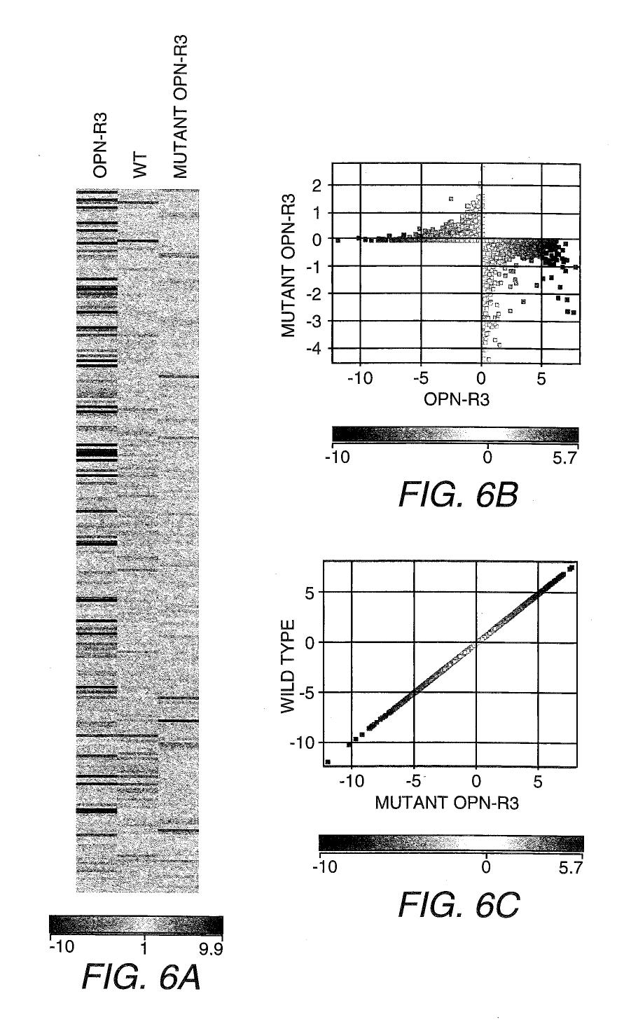

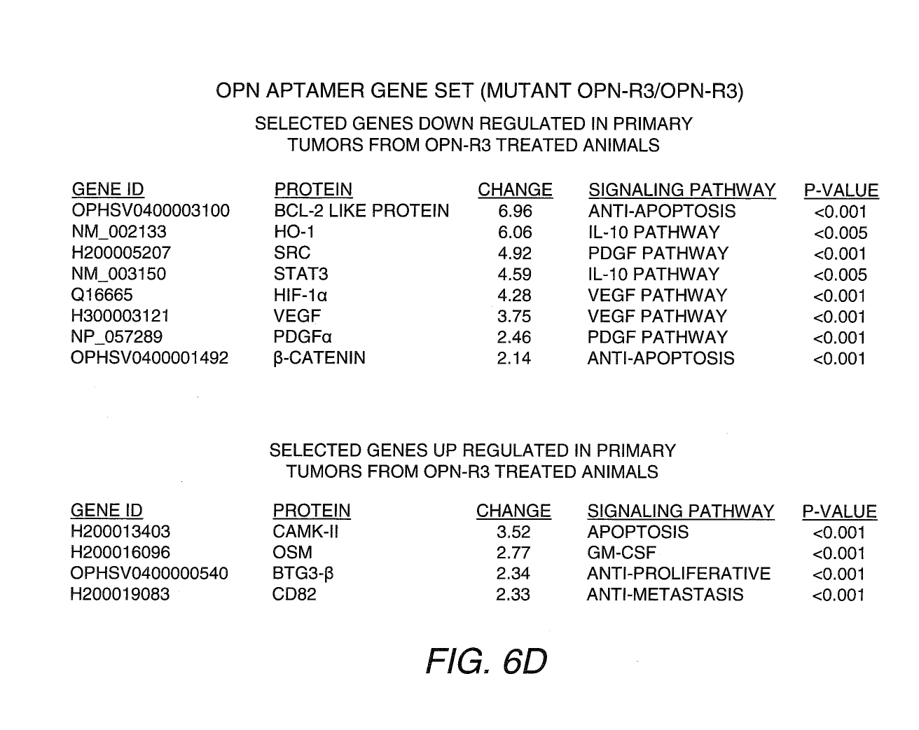

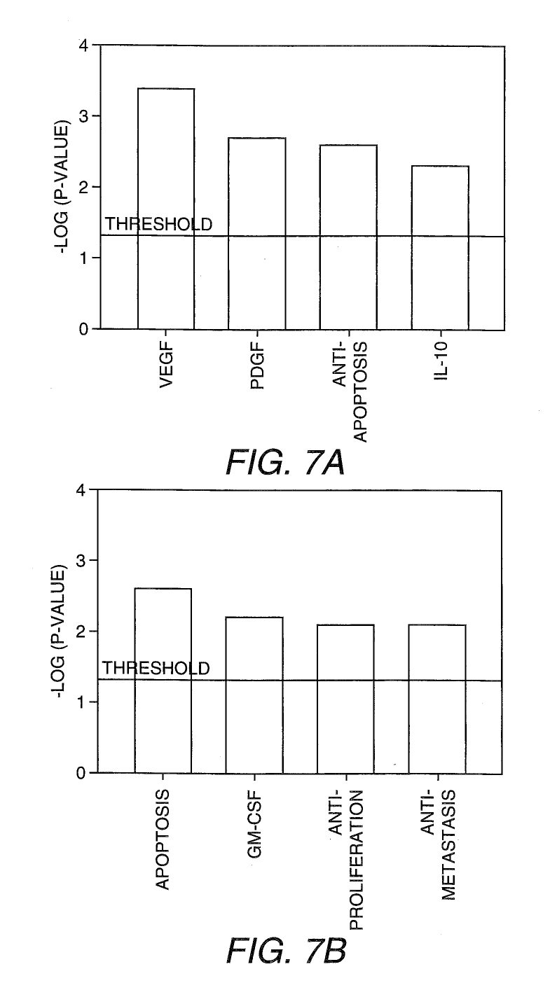

To identify the genes whose expression is regulated by OPN in the xenograft model of Example 4, RNA was extracted from primary tumors of wild-type (WT) animals and those treated with OPN-R3 and mutant OPN-R3. Total RNA was extracted from primary tumor using RNeasy mini kit (Qiagen, Valencia, Calif.). A total of nine animals were used (WT, n=3; OPN-R3, n=3; mutant OPN-R3, n=3). The cDNA synthesis, labeling, hybridization, and scanning were performed by the Duke University Microarray Facility. RNA was hybridized to the Human Operon v4.0 spotted array covering 35,000 human genes. The complete description of the array is available at wwvv.genome.duke.edu/cores/microarray. Samples from each animal were arrayed separately. Microarray data were analyzed by the Partek Genomics Suite software (Partek, St. Louis, Mo.). The reference set was defined to be the mean of the WT and mutant OPN-R3 groups. The heat map of the three groups and the scatter plots of WT versus mutant OPN-R3 and OPN-R3 versus mutant OPN-R3 are displayed in FIG. 6. The scatter plots indicate that significant differences in gene expression are present between the OPN-R3 and mutant OPN-R3 groups, while the WT and mutant OPN-R3 groups are not significantly different. The top eight genes down-regulated by >2-fold and the top four genes up-regulated by >2-fold in primary tumors from OPN-R3 treated animals are listed in FIG. 6D. Genes were then assigned to biological pathways using Ingenuity Pathway Analysis software (Ingenuity Systems, Redwood City, Calif.) (FIG. 7). The threshold value of −log (p-value) was set at 1.31, corresponding to a p-value of 0.05. This software suggested that OPN-R3 was associated with down-regulation of IL-10, VEGF, PDGF, and anti-apoptosis signaling with concomitant up-regulation of apoptosis, GM-CSF, anti-proliferative, and anti-metastasis signaling pathways.

Real-time RT-PCR and Western blot analysis were used to verify altered expression of the identified genes and proteins in OPN-R3 and mutant OPN-R3 groups. Real-time PCR was performed with the two-step reaction protocol using iQ SYBR Green detection kit (Bio-Rad Laboratories, Hercules, Calif.). First-strand cDNA was synthesized from 1 μg total RNA using the iScript Select cDNA synthesis kit (Bio-Rad Laboratories, Hercules, Calif.) at 48° C. for 30 minutes. Glyceraldehyde 3-phosphate dehydrogenase (GAPDH) was used as the endogenous control. The following primer sets were used for the quantitative PCR analysis.

GAPDH:
(SEQ ID NO: 20)
forward: 5′-AGCCTCAAGATCATCAGCAATGCC-3′
(SEQ ID NO: 21)
reverse: 5′-TGTGGTCATGAGTCCTTCCACGAT-3′
Hypoxia inducible factor-Iα (HIF-1α):
(SEQ ID NO: 22)
forward: 5′-GACTCAGCTATTCACCAAAG-3′
(SEQ ID NO: 23)
reverse: 5′-AAAGATATGATTGTGTCTCC-3′
VEGF:
(SEQ ID NO: 24)
forward: 5′-ATCACGAAGTGGTGAAGTTC-3′
(SEQ ID NO: 25)
reverse: 5′-AGGATGGCTTGAAGATGTAC-3
PDGFα:
(SEQ ID NO: 26)
forward: 5′-GACACCAGCCTGAGAGCTCA-3′
(SEQ ID NO: 27)
reverse: 5′-CCTGGTCTTGCAGACAGCGG-3′
SRC:
(SEQ ID NO: 28)
forward: 5′-GGCTGGAGGTCAAGCTGGGC-3′
(SEQ ID NO: 29)
reverse: 5′-GGAAGGCCTCTGGAGACATC-3′
β-Catenin:
(SEQ ID NO: 30)
forward: 5′-GTCCATGGGTGGGACACAGC-3′
(SEQ ID NO: 31)
reverse: 5′-CTGATAACAATTCGGTTGTG-3′
B-cell CLL/lymphoma-2 (BCL-2):
(SEQ ID NO: 32)
forward: 5′-GAGGTGATCCCCATGGCAGC-3′
(SEQ ID NO: 33)
reverse: 5′-TGTCCCTGGGGTGATGTGGA-3′
Heme-oxygenase-1 (HO-1):
(SEQ ID NO: 34)
forward: 5′-TGTACCACATCTATGTGGCC-3′
(SEQ ID NO: 35)
reverse: 5′-CCAGGTCCTGCTCCAGGGCA-3′
Signal Transducers and Activator of
Transcription 3 (STAT3):
(SEQ ID NO: 36)
forward: 5′-CAGCAGATGCTGGAGCAGCA-3′
(SEQ ID NO: 37)
reverse: 5′-CTTGAGGGTTTTATAGTTGA-3′
Oncostatin-M (OSM):
(SEQ ID NO: 38)
forward: 5′-GAAGCAGACAGATCTCATGC-3′
(SEQ ID NO: 39)
reverse: 5′-CTCCCTGCAGTGCTCTCTCA-3′
Calmodulin-dependent protein kinase-2A (CAMK2A):
(SEQ ID NO: 40)
forward: 5′-GGAAGCCAAGGATCTGATCA-3′
(SEQ ID NO: 41)
reverse: 5′-TGCATGCAGGATGCCACGGT-3′
B-cell translocation gene-3β (BTG3-β):
(SEQ ID NO: 42)
forward: 5′-AGGACAGGCCTACAGATGTA-3′
(SEQ ID NO: 43)
reverse: 5′-GAGAGTGAGCTCCTTTGGCA-3′
Cluster of Differentiation 82 (CD82):
(SEQ ID NO: 44)
forward: 5′-AGAGCAGTTTCATCTCTGTC-3′
(SEQ ID NO: 45)
reverse: 5′-GCAGCCCAGGAAGCCCATGA-3′

Real-time PCR parameters used were as follows: 95° C. for 3 minutes; 95° C. for 30 seconds, 55° C. for 35 seconds for 40 cycles; 95° C. for 1 minute, and 55° C. for 10 minutes. PCR was performed with iQ SYBR Green super mix, using the iCycler iQ Real-time PCR Detection System (Bio-Rad Laboratories, Hercules, Calif.). The 2-delta-delta Ct value was calculated following GAPDH normalization. Fold induction was determined relative to cells treated with mutant OPN-R3. A total of six animals were analyzed (OPN-R3, n=3; mutant OPN-R3, n=3). Data are representative of three experiments.

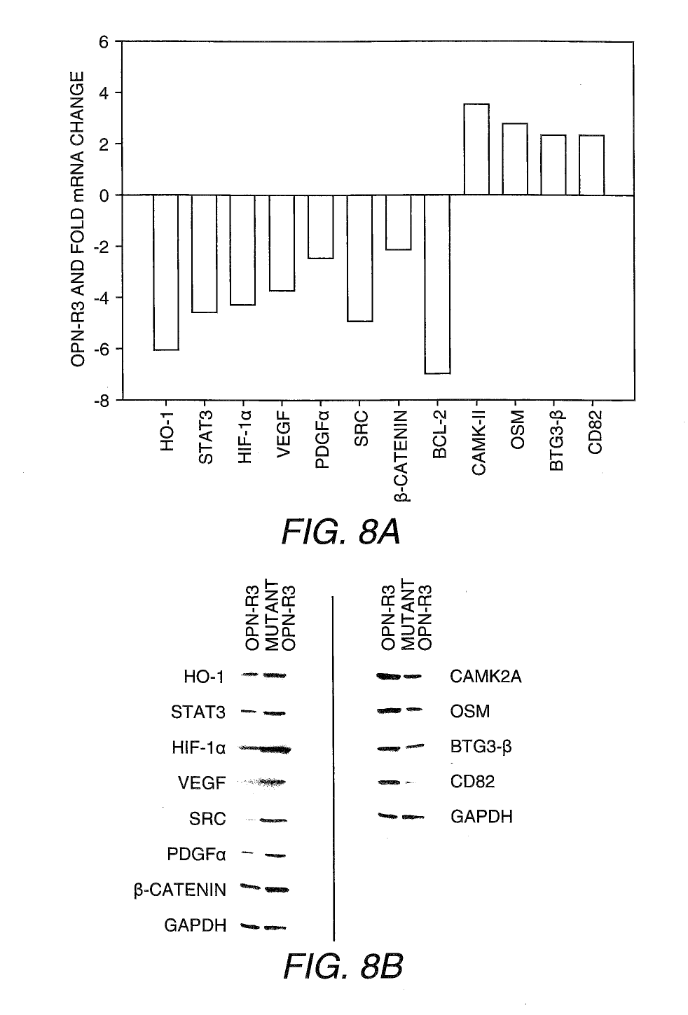

The RT-PCR results (FIG. 8A) corroborated the microarray results. The same pattern of gene expression changes was seen with mRNA levels as was seen with the microarray.

For Western blot analysis, primary tumor tissues were excited from OPN-R3 and mutant OPN-R3 aptamer-treated mice. Tumor tissues were lysed in buffer (0.8% NaCl, 0.02% KCl, 1% SDS, 10% Triton X-100, 0.5% sodium deoxycholic acid, 0.144% Na₂HPO₄, 0.024% KH₂PO₄, 2 mM phenylmethylsulfonyl fluoride, pH 7.4) and centrifuged at 12,000×g for 1 minutes at 4° C. The protein concentration was determined by the Bio-Rad protein assay kit. The protein samples were separated by 4-20% SDS-PAGE and electrotransferred onto polyvinylidene difluoride membranes (Amersham Biosciences, Piscataway, N.J. by semi-dry transfer. Blots are representative of three experiments.

Similar to the RT-PCR results, expression of the corresponding proteins was also altered in a fashion predicted by the microarray results (FIG. 8B). These results demonstrate that blockade of OPN binding through RNA aptamer targeting decreases expression of key proteins involved in the IL-10, VEGF, PDGF, and anti-apoptosis pathways with simultaneous increases in apoptosis, GM-CSF, anti-proliferative, and anti-metastasis signaling proteins.

The foregoing is illustrative of the present invention, and is not to be construed as limiting thereof. The invention is defined by the following claims, with equivalents of the claims to be included therein.

1-60. (canceled)

61. A polynucleotide aptamer that binds specifically to osteopontin.

62. The polynucleotide aptamer of claim 61, wherein said aptamer binds specifically to both human osteopontin and mouse osteopontin.

63. The polynucleotide aptamer of claim 61, wherein said aptamer binds to osteopontin with a Kd of less than about 1000 nM.

64. The polynucleotide aptamer of claim 61, which consists of about 10 to about 100 nucleotides.

65. The polynucleotide aptamer of claim 61, which is a RNA aptamer.

66. The polynucleotide aptamer of claim 65, comprising a nucleotide sequence at least 70% identical to any one of SEQ ID NOS: 1-14 or a fragment thereof of at least ten contiguous nucleotides.

67. The polynucleotide aptamer of claim 66, comprising the nucleotide sequence of any one of SEQ ID NOS: 1-14 or a fragment thereof of at least ten contiguous nucleotides.

68. The polynucleotide aptamer of claim 66, consisting of the nucleotide sequence of any one of SEQ ID NOS: 1-14 or a fragment thereof of at least ten contiguous nucleotides.

69. A polynucleotide encoding the polynucleotide aptamer of claim 61.

70. A vector comprising the polynucleotide of claim 69.

71. A cell comprising the polynucleotide aptamer of claim 61.

72. A pharmaceutical composition comprising the polynucleotide aptamer of claim 61 and a pharmaceutically acceptable carrier.

73. A method of inhibiting at least one biological function of osteopontin, comprising contacting osteopontin with the polynucleotide aptamer of claim 61 in an amount effective to inhibit at least one biological function.

74. A method of inhibiting the adhesion, migration, or invasion ability of a cell, comprising contacting said cell with the polynucleotide aptamer of claim 61 in an amount effective to inhibit the adhesion, migration, or invasion ability of said cell.

75. A method of treating a disease or disorder associated with osteopontin in a subject, comprising administering to said subject the polynucleotide aptamer of claim 61 in an amount effective to treat the disease or disorder.

76. A method of treating cancer in a subject, comprising administering to said subject the polynucleotide aptamer of claim 61 in an amount effective to treat cancer.

77. A method of inhibiting tumor metastasis in a subject, comprising administering to said subject the polynucleotide aptamer of claim 61 in an amount effective to inhibit tumor metastasis.

78. A method of promoting wound healing and/or inhibiting scar formation in a subject, comprising administering to said subject the polynucleotide aptamer of claim 61 in an amount effective to promote wound healing and/or inhibit scar formation.

79. A method of measuring the level of osteopontin in a subject, comprising binding osteopontin with the polynucleotide aptamer of claim 61 and determining the amount of aptamer bound to osteopontin.

80. A method of diagnosing a disease or disorder associated with osteopontin in a subject, comprising measuring the level of osteopontin in the subject by binding osteopontin with the polynucleotide aptamer of claim 61 and determining the amount of aptamer bound to osteopontin.

81. A method of inducing apoptosis in a cell of a subject, comprising administering to said subject the polynucleotide aptamer of claim 61 in an amount effective to induce apoptosis.

82. A method of inhibiting angiogenesis and/or vascularization in a subject, comprising administering to said subject the polynucleotide aptamer of claim 61 in an amount effective to inhibit angiogenesis and/or vascularization.
